(19) United States
(12) Patent Application Publication
(10) Pub. No.: US 2023/0137271 A1
(43) Pub. Date: May 4, 2023

(54) COMPUTER ARCHITECTURE FOR GENERATING A REFERENCE DATA TABLE

(71) Applicant: GUARDANT HEALTH, INC., Palo Alto, CA (US)

(72) Inventors: Naveen KUMAR, Redwood City, CA (US); Jingwen ZHANG, Killeen, TX (US); Nisha SUBRAMANIAN, Redwood City, CA (US); Gautam NAYAK, Fremont, CA (US); David HANNA, Palo Alto, CA (US); Shunxin LU, Dublin, CA (US)

(21) Appl. No.: 17/937,050

(22) Filed: Sep. 30, 2022

Related U.S. Application Data
(63) Continuation-in-part of application No. PCT/US2022/042262, filed on Aug. 31, 2022.
Application No. PCT/US2022/042262 is a continuation-in-part of application No. PCT/US2022/038941, filed on Jul. 29, 2022.
Application No. PCT/US2022/038941 is a continuation-in-part of application No. PCT/US2022/032250, filed on Jun. 3, 2022.
(60) Provisional application No. 63/250,912, filed on Sep. 30, 2021.

(51) Int. Cl. G16H 20/10; G16H 50/70

(52) U.S. Cl. CPC G16H 20/10; G16H 50/70

(57) ABSTRACT

An integrated data repository may be generated that includes genomics information and health insurance claims data information for a common group of individuals. The health insurance claims data can be analyzed to determine insurance code identifiers that correspond to treatments provided to individuals in which a biological condition is present. The insurance code identifiers can be used to query one or more databases to obtain additional information about the treatments. A treatment reference table can be generated using the insurance claims data and the additional information obtained from the one or more databases. The treatment reference table can be used to identify cohorts of individuals that received one or more treatments in relation to the biological condition and also to determine one or more features of the individuals included in the cohorts.

### CROSS-REFERENCE TO RELATED APPLICATION

This patent application claims the benefit of priority to U.S. Provisional Application Ser. No. 63/250,912, filed on Sep. 30, 2021, and is a Continuation-in-Parts of PCT Application No. PCT/US2022/032250 filed Jun. 3, 2022, and entitled “Computer Architecture for Generating an Integrated Data Repository,” and is a Continuation-in-Parts of PCT Application No. PCT/US2022/038941 filed Jul. 29, 2022, and entitled “Computer Architecture for Identifying Lines of Therapy,” and is a Continuation-in-Parts of PCT Application No. PCT/US2022/042262 filed Aug. 31, 2022, and entitled “Data Repository, System and Method for Cohort Selection,” the entire contents of which are each incorporated by reference herein in their entirety.

### TECHNICAL FIELD

Implementations of the present disclosure relate generally to the field of computer architecture, and more particularly to implementations of computer architectures for generating a reference data table that indicates identifiers of treatments provided to patients in which one or more biological conditions may be present.

### BACKGROUND

As individuals visit healthcare providers to treat one or more biological conditions, various types of documentation may be generated. For example, medical records may be produced by healthcare providers that include clinical observations recorded by a healthcare provider, laboratory test results, diagnostic test information, imaging information, dental health information, one or more combinations thereof, and the like. Additionally, billing records may be generated that indicate payment information with respect to at least one of products or services provided to individuals by healthcare providers. Further, health insurance claims information may be generated that indicates information obtained by health insurance companies related to the treatment of individuals with respect to one or more biological conditions.

### BRIEF DESCRIPTION OF THE DRAWINGS

FIG. 1 illustrates an example architecture to generate an integrated data repository that includes multiple types of healthcare data and to generate a reference data table that indicates identifiers of treatments provided to patients in which one or more biological conditions may be present, according to one or more implementations.

FIG. 2 illustrates an example framework corresponding to an arrangement of data tables in an integrated data repository, according to one or more implementations.

FIG. 3 illustrates an architecture to generate one or more datasets from information retrieved from a data repository that integrates health related data from a number of sources, according to one or more implementations.

FIG. 4 illustrates an architecture to generate an integrated data repository that includes de-identified health insurance claims data and de-identified genomics data, according to one or more implementations.

FIG. 5 illustrates a framework to generate a dataset, by a data pipeline system, based on data stored by an integrated data repository, according to one or more implementations.

FIG. 6 illustrates an architecture to generate a reference data table indicating identifiers of treatments provided to patients in which one or more biological conditions may be present, according to one or more implementations.

FIG. 7 is a flow diagram of an example process to generate a treatment reference table that includes information about treatments provided to patients in which one or more biological conditions may be present, according to one or more implementations.

FIG. 8 is a flow diagram of an example process to determine an identifier of a drug that corresponds to an insurance code identifier using one or more application programming interface (API) requests, according to one or more implementations.

FIG. 9 is a flow diagram of an example process to determine a class corresponding to an identifier of a treatment and to include information related to the class in a reference data table that includes the identifier of the treatment, according to one or more implementations.

FIG. 10 illustrates a diagrammatic representation of a machine in the form of a computer system within which a set of instructions may be executed for causing the machine to perform any one or more of the methodologies discussed herein, according to one or more implementations.

### DETAILED DESCRIPTION

The following description and the drawings sufficiently illustrate specific implementations to enable those skilled in the art to practice them. Other implementations may incorporate structural, logical, electrical, process, and other changes. Portions and features of some implementations may be included in, or substituted for, those of other implementations. Implementations set forth in the claims encompass all available equivalents of those claims.

The analysis of healthcare data using existing systems and techniques is typically performed with respect to medical records generated by healthcare providers. As used herein, a healthcare provider may refer to an entity, individual, or group of individuals involved in providing care to individuals in relation to at least one of the treatment or prevention of one or more biological conditions. In addition, as used herein, a biological condition can refer to an abnormality of function and/or structure in an individual to such a degree as to produce or threaten to produce a detectable feature of the abnormality. A biological condition can be characterized by external and/or internal characteristics, signs, and/or symptoms that indicate a deviation from a biological norm in one or more populations. A biological condition can include at least one of one or more diseases, one or more disorders, one or more injuries, one or more syndromes, one or more disabilities, one or more infections, one or more isolated symptoms, or other atypical variations of biological structure and/or function of individuals. Additionally, a treatment, as used herein, can refer to a substance, procedure, routine, device, and/or other intervention that can administered or performed with the intent of alleviating one or more effects of a biological condition in an individual. In one or more examples, a treatment may include a substance that is metabolized by the individual. The substance may include a composition of matter, such as a pharmaceutical composition. The substance may be delivered to the individual via a number of methods, such as ingestion, injection, absorption, or inhalation. A treatment may also include physical interventions, such as one or more surgeries.

The healthcare data typically analyzed by existing systems includes unstructured data. Unstructured data can include data that is not organized according to a pre-defined or standardized format. For example, unstructured data may include notes made by a healthcare provider that is comprised of free text. That is, the manner in which the notes are captured does not include pre-defined inputs that are selectable by the healthcare provider, such as via a drop-down menu or via a list. Rather, the notes include text entered by a healthcare provider that may include sentences, sentence fragments, words, letters, symbols, abbreviations, one or more combinations thereof, and so forth. In some cases, unstructured data may be partially structured. For example, a provider could select an insurance billing code from a predefined list of insurance billing codes, and add unstructured notes to data associated with that billing code.

Existing systems typically devote a large amount of computing resources to analyzing unstructured data in order to extract information that may be relevant to analyses being performed by the existing systems. In some cases, existing systems may analyze unstructured data and transform the unstructured data to a structured format in order to facilitate the analysis of the previously unstructured data. The analysis of unstructured data by existing systems can be inefficient as well as inaccurate. In scenarios where the unstructured data is obtained from healthcare data, the importance of accurately analyzing the information is high because the analysis may be related to at least one of the treatment or diagnosis of a number of individuals with respect to one or more biological conditions. Thus, inaccurate analyses of healthcare data may have a detrimental impact on the health of individuals.

The implementations of techniques, architectures, frameworks, systems, processes, and computer-readable instructions described herein are directed to analyzing health insurance claims data to derive information about at least one of the health or treatment of individuals. In contrast to existing systems, health insurance claims data is structured according to one or more formats and stored by a number of data tables. The data tables may include codes or other alphanumeric information indicating treatments received by individuals, dates of treatments, dosage information, diagnoses of individuals with respect to one or more biological conditions, information related to visits to healthcare providers, dates of visits to healthcare providers, billing information, and the like. The implementations described herein may be used to accurately analyze health insurance claims data for hundreds, up to thousands of individuals in which one or more biological conditions are present. In various examples, tens of thousands, hundreds of thousands, up to millions of rows and/or columns of health insurance claims data may be analyzed to determine health-related information for individuals in which one or more biological conditions are present.

In various examples, the implementations described herein can integrate molecular data with health insurance claims data. The molecular data may include information derived from tissue samples extracted from a number of individuals. In addition, the molecular data may include information derived from blood samples extracted from a number of individuals. In one or more illustrative examples, the molecular data may include genomics data. Further, in one or more examples, the health insurance claims data may be integrated with germline genetic information for a number of individuals.

An integrated data repository may be created that combines the health insurance claims data for individuals with the molecular data of the individuals. In one or more examples, an identifier may be generated for an individual that is associated with both the health insurance claims data of the individual and the molecular data of the individual. Both the molecular data and the health insurance claims data stored by the integrated data repository may be accessible using a single identifier of the individual. In one or more illustrative examples, the identifier for an individual may include an encrypted security key. In various examples, the integrated data repository may include a number of data tables corresponding to different aspects of the data stored within the data repository. For example, a first data table can be generated that includes summary data of individuals included in the integrated data repository, such as personal information, and a second data table may be generated that includes data corresponding to visits to healthcare providers. Additionally, a third data table may be generated indicating medical procedures provided to individuals and a fourth data table may be generated indicating information related to prescriptions obtained by individuals. Further, a fifth data table may be generated that includes molecular information of individuals.

The data tables included in the integrated data repository may be connected via logical links. In this way, a query to retrieve information from one data table may cause information from one or more additional data tables to be retrieved. Information stored by the linked data tables may be accessed to generate a number of different datasets that may be used to analyze the information stored by the integrated data repository. In one or more examples, the information stored by the integrated data repository can be analyzed to extract biological meaning with regard to a patient or a group of patients. Additionally, the information stored by the integrated data repository can be analyzed to determine a biological state of individuals. The biological state can correspond to determining whether a biological condition is present or not with respect to a patient or a group of patients.

In one or more further examples, the information stored by the integrated data repository may be analyzed by one or more algorithms to generate datasets that are organized according to one or more schemas. The datasets may indicate treatment received by an individual over a period of time with respect to a biological condition. The datasets may also indicate cohorts of individuals included in the integrated data repository having a number of common characteristics. In various examples, the datasets may consolidate and arrange information from a number of different data sources, including the integrated data repository. The datasets may be analyzed with respect to a number of queries to indicate information that may be of interest to at least one of healthcare providers, patients, or providers of treatments of biological conditions. For example, one or more datasets may be integrated and analyzed to determine a survival rate of individuals in which a biological condition is present and having a specified genomic profile in response to receiving a specified treatment.

The implementations described herein may provide a platform to integrate health insurance claims data and molecular data for individuals that is not found in existing systems that typically rely on electronic medical records that include an amount of unstructured data. By generating and analyzing structured health insurance claims data that has been integrated with molecular data, the implementations described herein may provide more accurate characterizations of the integrated data in relation to existing systems that rely on relatively inaccurate, unstructured electronic medical records data. Additionally, implementations described herein generate analytics ready datasets that enable the analysis of health information about individuals in a confidential and anonymized manner.

Additionally, insurance claims data can include information that can be used to determine at least one of one or more biological conditions present in individuals, treatments provided to individuals in which one or more biological conditions are present, a timeline of treatments provided to individuals, or modifications to biological conditions present in individuals. Insurance claims data in its raw form, however, can be difficult to interpret. Further, large amounts of insurance claims data can be generated for an individual over a period of time relating to one or more treatments provided to an individual based on a biological condition being present in the individual. In one or more examples, hundreds of insurance claims up to thousands of insurance claims or more can be generated over a period of time based on one or more treatments provided to the individual with respect to a biological condition. Also, it can be challenging to relate one piece of insurance claims data for an individual with another piece of insurance claims data for the individual with respect to a given biological condition because the insurance claims data is typically a series of codes that do not provide any indication as to how the codes are related or what the codes themselves mean. Thus, determining tangible insights, trends, correlations, and the like, related to treatments that one individual receives for one or more biological conditions using insurance claims data is not straightforward and involves the analysis of large amounts of disparate data. In situations where insurance claims data for multiple individuals is analyzed, the complexity, time, and computing resources used to determine relationships, correlations, insights, and so forth with respect to the treatments of the individuals increases at a large rate, even exponentially.

The techniques, systems, architectures, frameworks, processes, and methods described herein are also directed to generating a reference data table that can be used to analyze insurance claims data. In one or more examples, the reference data table can indicate information about one or more treatments that are provided to individuals in which a biological condition is present. To illustrate, the reference data table can include information about one or more drugs provided to individuals in which a biological condition is present. In various examples, the reference data table can indicate, for a given insurance code identifier, a name of a treatment, a class of a treatment, one or more ingredients included in the treatment, a source of information about the treatment, one or more additional identifiers of the treatment, one or more combinations thereof, and the like.

In one or more implementations, insurance code identifiers can be extracted from one or more data tables that include a number of insurance code identifiers related to one or more treatments of individuals in which a biological condition is present. The insurance code identifiers can be analyzed with respect to one or more criteria in relation to at least one format of insurance code identifiers. For example, the insurance code identifiers can be analyzed to determine whether the insurance code identifiers correspond to a National Drug Code (NDC) format. In one or more illustrative examples, the insurance code identifiers can be analyzed to determine whether the insurance code identifiers correspond to an NDC 9 format, an NDC 10 format, an NDC 11 format, or another NDC format. In one or more implementations, application programming interface (API) requests can be generated that query a data repository to determine updated insurance code identifiers that correspond to earlier versions of insurance code identifiers. In at least some examples, responses to the API requests can include NDC codes having a format that is different from the format of the NDC codes used to generate the API requests. To illustrate, an API request can be generated and sent to a data repository management system, where the API request can include a modified version of an insurance code identifier that initially had an NDC 9 format or an NDC 10 format. In these scenarios, a response to the request can be a data file that includes a version of the insurance code identifier that corresponds to an NDC 11 format. Insurance code identifiers that correspond to an NDC 11 format can be used to generate additional API requests to retrieve information from one or more additional datasets stored by a data repository management system. The responses to the additional API requests can include at least one of an identifier of the treatment within the database management system, one or more names of the treatment, information related to one or more classes of the treatment, or information related to one or more sources of the one or more classes of the treatment.

A reference data table can be generated using at least a portion of the information obtained from responses to API requests that retrieve information from one or more datasets. In one or more examples, a respective row can be created in the reference data table for individual treatments that have valid insurance code identifiers. Columns of the reference table can be populated using information that corresponds to individual treatments where the information is obtained from API requests to obtain data included in one or more datasets. For example, the reference data table can include one or more columns that indicate at least one of an original NDC code obtained from insurance claims data, an identifier of a treatment that corresponds to the original NDC code, a name of a treatment that corresponds to the original NDC code, or a class of a treatment that corresponds to the original NDC code.

In one or more illustrative examples, an analysis can be performed to determine an effectiveness of a treatment with respect to individuals having one or more specified genetic mutations. To determine the effectiveness of the treatment, insurance claims data can be analyzed that indicates treatments provided to individuals. The reference table can be used to determine treatments provided to individuals by analyzing the insurance code identifiers included in the insurance claims data in relation to the insurance code identifiers and treatments included in the reference table. Subsequently, a cohort of individuals receiving the treatment can be identified by using the reference table to determine the insurance code identifier that corresponds to the treatment and then determining individuals having insurance claims data that includes the insurance code identifier. An additional analysis can then be performed with respect to the cohort of individuals to determine the effectiveness of the treatment provided to the individuals based on a number of additional criteria. For example, genetic material included in at least one of blood or tissue samples can be analyzed to determine whether individuals that exhibit a specific tumor receiving the treatment are increasing in size or decreasing in size.

As a result, the reference data table can include a row for individual NDC codes included in the insurance claims data and indicate information related to treatments that correspond to the individual NDC codes. In this way, the insurance claims data can be analyzed with respect to the treatments provided to the individuals included in the insurance claims data. In the absence of the reference data table, each time that insurance claims data is to be analyzed with respect to treatments obtained by individuals, the datasets that include treatment information would be queried and analyzed for validity. This is a time-consuming, computing resource intensive, and inefficient endeavor. Thus, generating a reference data table that indicates insurance code identifiers that correspond to respective treatments and that is readily accessible, reduces the number of computing resources utilized and the time used to perform the analysis in relation to situations without the reference data table where the correlations between individual insurance code identifiers and respective treatments are determined for each analysis.

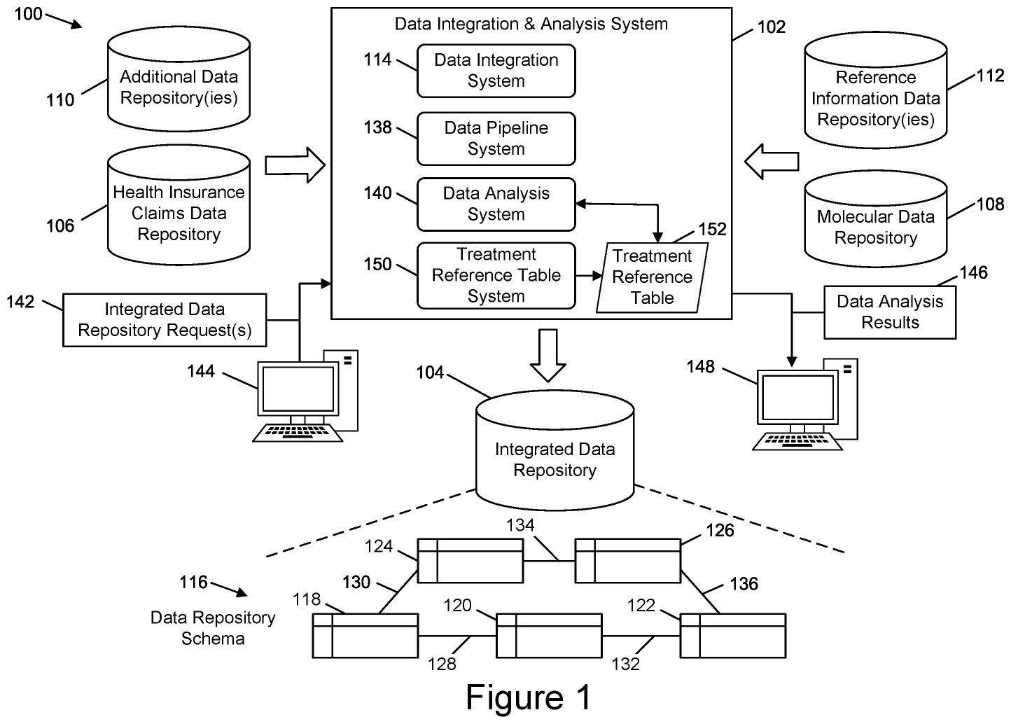

FIG. 1 illustrates an example architecture 100 to generate an integrated data repository that includes multiple types of healthcare data, according to one or more implementations. The architecture 100 may include a data integration and analysis system 102. The data integration and analysis system 102 may obtain data from a number of data sources and integrate the data from the data sources into an integrated data repository 104. For example, the data integration and analysis system 102 may obtain data from a health insurance claims data repository 106. In various examples, the data integration and analysis system 102 and the health insurance claims data repository 106 may be created and maintained by different entities. In one or more additional examples, the data integration and analysis system 102 and the health insurance claims data repository 106 may be created and maintained by a same entity.

The data integration and analysis system 102 may be implemented by one or more computing devices. The one or more computing devices can include one or more server computing devices, one or more desktop computing devices, one or more laptop computing devices, one or more tablet computing devices, one or more mobile computing devices, or combinations thereof. In certain implementations, at least a portion of the one or more computing devices can be implemented in a distributed computing environment. For example, at least a portion of the one or more computing devices can be implemented in a cloud computing architecture. In scenarios where the computing systems used to implement the data integration and analysis system 102 are configured in a distributed computing architecture, processing operations may be performed concurrently by multiple virtual machines. In various examples, the data integration and analysis system 102 may implement multithreading techniques. The implementation of a distributed computing architecture and multithreading techniques cause the data integration and analysis system 102 to utilize fewer computing resources in relation to computing architectures that do not implement these techniques.

The health insurance claims data repository 106 may store information obtained from one or more health insurance companies that corresponds to insurance claims made by subscribers of the one or more health insurance companies. The health insurance claims data repository 106 may be arranged (e.g., sorted) by patient identifier. The patient identifier may be based on the patient's first name, last name, date of birth, social security number, address, employer, and the like. The data stored by the health insurance claims data repository 106 may include structured data that is arranged in one or more data tables. The one or more data tables storing the structured data may include a number of rows and a number of columns that indicate information about health insurance claims made by subscribers of one or more health insurance companies in relation to procedures and/or treatments received by the subscribers from healthcare providers. At least a portion of the rows and columns of the data tables stored by the health insurance claims data repository 106 may include health insurance codes that may indicate diagnoses of biological conditions, and treatments and/or procedures obtained by subscribers of the one or more health insurance companies. In various examples, the health insurance codes may also indicate diagnostic procedures obtained by individuals that are related to one or more biological conditions that may be present in the individuals. In one or more examples, a diagnostic procedure may provide information used in the detection of the presence of a biological condition. A diagnostic procedure may also provide information used to determine a progression of a biological condition. In one or more illustrative examples, a diagnostic procedure may include one or more imaging procedures, one or more assays, one or more laboratory procedures, one or more combinations thereof, and the like.

The data integration and analysis system 102 may also obtain information from a molecular data repository 108. The molecular data repository 108 may store data of a number of individuals related to genomic information, genetic information, metabolomic information, transcriptomic information, fragmentiomic information, immune receptor information, methylation information, epigenomic information, and/or proteomic information, Immunohistochemistry (IHC), and immunofluorescence (IF). In one or more examples, the data integration and analysis system 102 and the molecular data repository 108 may be created and maintained by different entities. In one or more additional examples, the data integration and analysis system 102 and the molecular data repository 108 may be created and maintained by a same entity. As used herein, “fragmentomic information” may include, among other things, information related to the analysis of the length of DNA or RNA fragments to determine the presence or absence of a tumor and to determine characteristics of the tumors. In one or more illustrative examples, the fragmentiomic information can correspond to nucleosomal structure and transcription factor binding sites.

The genomic information may indicate one or more mutations corresponding to genes of the individuals. A mutation to a gene of individuals may correspond to differences between a sequence of nucleic acids of the individuals and one or more reference genomes. The reference genome may include a known reference genome, such as hg19. In various examples, a mutation of a gene of an individual may correspond to a difference in a germline gene of an individual in relation to the reference genome. In one or more additional examples, the reference genome may include a germline genome of an individual. In one or more further examples, a mutation to a gene of an individual may include a somatic mutation. Mutations to genes of individuals may be related to insertions, deletions, single nucleotide variants, loss of heterozygosity, duplication, amplification, translocation, fusion genes, or one or more combinations thereof. In at least some examples, the genomic information can correspond to non-coding regions of a genome. The non-coding regions can be related to the regulation of one or more genes. In one or more examples, the analysis of the non-coding regions can detect one or more epigenetic signatures of one or more patients.

In one or more illustrative examples, genomic information stored by the molecular data repository 108 may include genomic profiles of tumor cells present within individuals. In these situations, the genomic information may be derived from an analysis of genetic material, such as deoxyribonucleic acid (DNA) and/or ribonucleic acid (RNA), found in blood samples of individuals that is present due to the degradation of tumor cells present in the individuals. In one or more examples, the genomic information of tumor cells of individuals may correspond to one or more target regions. One or more mutations present with respect to the one or more target regions may indicate the presence of tumor cells in individuals. The genomic information stored by the molecular data repository 108 may be generated in relation to an assay or other diagnostic test that may determine one or more mutations with respect to one or more target regions of the reference genome.

In one or more illustrative examples, the genetic material can be derived from a sample, including, but not limited to, a tissue sample or tumor biopsy, circulating tumor cells (CTCs), exosomes or efferosomes, or from circulating nucleic acids. In various examples, the circulating nucleic acids can be referred to herein as “cell-free DNA.” “Cell-free DNA,” “cfDNA molecules,” or simply “cfDNA” include DNA molecules that occur in a subject in extracellular form (e.g., in blood, serum, plasma, or other bodily fluids such as lymph, cerebrospinal fluid, urine, or sputum) and includes DNA not contained within or otherwise bound to a cell at the point of isolation from the subject. While the DNA originally existed in a cell or cells of a large complex biological organism (e.g., a mammal) or other cells, such as bacteria, colonizing the organism, the DNA has undergone release from the cell(s) into a fluid found in the organism. cfDNA includes, but is not limited to, cell-free genomic DNA of the subject (e.g., a human subject's genomic DNA) and cell-free DNA of microbes, such as bacteria, inhabiting the subject (whether pathogenic bacteria or bacteria normally found in commonly colonized locations such as the gut or skin of healthy controls), but does not include the cell-free DNA of microbes that have merely contaminated a sample of bodily fluid. Typically, cfDNA may be obtained by obtaining a sample of the fluid without the need to perform an in vitro cell lysis step and also includes removal of cells present in the fluid (e.g., centrifugation of blood to remove cells).

In one or more additional examples, the data integration and analysis system 102 may obtain information from one or more additional data repositories 110. The one or more additional data repositories 110 may store data related to electronic medical records of individuals for which data is present in at least one of the health insurance claims data repository 106 or the molecular data repository 108. Further, the one or more additional data repositories 110 may store data related to pathology reports of individuals for which data is present in at least one of the health insurance claims data repository 106 or the molecular data repository 108. In various examples, the one or more additional data repositories 110 may store data related to biological conditions and/or treatments for biological conditions. In one or more examples, the data integration and analysis system 102 and at least a portion of the one or more additional data repositories 110 may be created and maintained by different entities. In one or more further examples, the data integration and analysis system 102 and at least a portion of the one or more additional data repositories 110 may be created and maintained by a same entity.

In one or more further implementations, the data integration and analysis system 102 may obtain information from one or more reference information data repositories 112. The one or more reference information data repositories 112 may store information that includes definitions, standards, protocols, vocabularies, one or more combinations thereof, and the like. In various examples, the information stored by the one or more reference information data repositories may correspond to biological conditions and/or treatments for biological conditions. In one or more illustrative examples, the one or more reference information data repositories 112 may include RxNorm. (RxNorm provides normalized names for clinical drugs and links its names to many of the drug vocabularies used in pharmacy management and drug interaction software.) In one or more examples, the data integration and analysis system 102 and at least a portion of the one or more reference information data repositories 112 may be created and maintained by different entities. In one or more further examples, the data integration and analysis system 102 and at least a portion of the one or more reference information data repositories 112 may be created and maintained by a same entity.

The data integration and analysis system 102 may obtain data from at least one of the health insurance claims data repository 106, the molecular data repository 108, the one or more additional data repositories 110, or the reference information data repositories 112 via one or more communication networks accessible to the data integration and analysis system 102 and accessible to at least one of the health insurance claims data repository 106, the molecular data repository 108, the one or more additional data repositories 110, or the reference information data repositories 112. The data integration and analysis system 102 may also obtain data from at least one of the health insurance claims data repository 106, the molecular data repository 108, the one or more additional data repositories 110, or the reference information data repositories 112 via one or more secure communication channels. In addition, the data integration and analysis system 102 may obtain data from at least one of the health insurance claims data repository 106, the molecular data repository 108, the one or more additional data repositories 110, or the reference information data repositories 112 via one or more calls of an application programming interface (API).

The data integration and analysis system 102 may include a data integration system 114. The data integration system 114 may obtain data from the health insurance claims data repository 106 and the molecular data repository 108 to generate the integrated data repository 104. The data integration system 114 may also obtain data from the one or more additional data repositories 110 to generate the integrated data repository 104. In various examples, the data integration system 114 may implement one or more natural language processing techniques to integrate data from the one or more additional data repositories 110 into the integrated data repository 104.

In one or more examples, the data integration system 114 may generate one or more tokens to identify individuals that have data stored in the health insurance claims data repository 106 and that have data stored in the molecular data repository 108. In various examples, the data integration system 114 may generate one or more tokens by implementing one or more hash functions. The data integration system 114 may implement the one or more hash functions to generate the one or more tokens based on information stored by at least one of the health insurance claims data repository 106 or the molecular data repository 108. For example, the information used by the data integration system 114 to generate individual tokens by implementing a hash function may include at least one of an identifier of respective individuals, date of birth of the respective individuals, a postal code of the respective individuals, date of birth of the respective individuals, or a gender of the respective individuals. In one or more illustrative examples, the identifiers of the respective individuals may include a combination of at least a portion of a first name of the respective individuals and at least a portion of the last name of the respective individuals. Tokens generated using data from different data repositories may correspond to the same or similar information or the same or similar type stored by the different data repositories. To illustrate, tokens may be generated using a portion of names of individuals, date of birth, at least a portion of a postal code, and gender obtained from the health insurance claims data repository 106 and the molecular data repository 108.

The data integration system 114 may integrate data from a number of different data sources by analyzing tokens generated by implementing one or more hash functions using data obtained from the number of different data sources. For example, the data integration system 114 may obtain one or more first tokens generated from data stored by the health insurance claims data repository 106 and one or more second tokens generated from data stored by the molecular data repository 108. The data integration system 114 may analyze the one or more first tokens with respect to the one or more second tokens to determine individual first tokens that correspond to individual second tokens. In one or more illustrative examples, the data integration system 114 may identify individual first tokens that match individual second tokens. A first token may match a second token when the data of the first token has at least a threshold amount of similarity with respect to the data of the second token. In one or more examples, a first token may match a second token when the data of the first token is the same as the data of the second token. To illustrate, a first token may match a second token when an alphanumeric string of the first token is the same as an alphanumeric string of the second token.

By determining a first token generated using data stored by the health insurance claims data repository 106 that corresponds to a second token generated using data stored by the molecular data repository 108, the data integration system 114 may identify an individual having data that is stored in both the health insurance claims data repository 106 and in the molecular data repository 108. In this way, the data integration system 114 may obtain data from the health insurance claims data repository 106 from a number of individuals and data from the molecular data repository 108 from the same number of individuals and store the health insurance claims data and the molecular data for the number of individuals in the integrated data repository 104.

The data integration system 114 may also integrate data stored by the one or more additional data repositories 110 with data from the health insurance claims data repository 106 and the molecular data repository 108 to generate the integrated data repository 104. To illustrate, the data integration system 114 may obtain one or more third tokens generated from data stored by an additional data repository 110, such as a data repository storing data corresponding to pathology reports. The data integration system 114 may analyze the one or more third tokens with respect to the first tokens generated using information stored by the health insurance claims data repository 106 and the second tokens generated using information stored by the molecular data repository 108 to determine respective third tokens that correspond to individuals first tokens and individual second tokens. In one or more illustrative examples, the data integration system 114 may identify third tokens generated using one or more hash functions and a common set of information obtained from the health insurance claims data repository 106, the molecular data repository 108, and the additional data repository 110.

By determining a third token generated using data stored by an additional data repository 110 that corresponds to a first token generated using data stored by the health insurance claims data repository 106 and a second token generated using data stored by the molecular data repository 108, the data integration system 114 may identify an individual having data that is stored in the health insurance claims data repository 106, the molecular data repository 108, and in an additional data repository 110. In this way, the data integration system 114 may obtain data from the health insurance claims data repository 106 from a number of individuals and data from the molecular data repository 108 and an additional data repository 110 from the same number of individuals and store the health insurance claims data, the molecular data, and the additional data for the number of individuals in the integrated data repository 104.

The data stored by the integrated data repository 104 for the number of individuals may be accessible using respective identifiers of individuals. The data integration system 114 may implement a number of techniques as part of a de-identification process with respect to storing and retrieving information of individuals in the integrated data repository 104. The identifiers of individuals may correspond to keys that are generated using at least one hash function. The identifiers of the individuals may also be generated by implementing one or more salting processes with respect to the keys generated using the at least one hash function. the tokens generated using one or more hash functions and a common set of information obtained from the health insurance claims data repository 106, the molecular data repository 108, and/or the additional data repository 110. In one or more illustrative examples, the identifiers generated by the data integration system 114 to access information for respective individuals that is stored by the integrated data repository 104 may be unique for each individual. In one or more examples, the identifiers of the individuals may be generated using at least a portion of the information used to generate the tokens related to the individuals. In one or more additional examples, the identifiers of the individuals may be generated using different information from the information used to generate the tokens related to the individuals.

The data integration system 114 may also generate the integrated data repository 104 from a number of different combinations of data repositories in a similar manner. For example, the data integration system 114 may obtain tokens generated from information stored by the health insurance claims data repository 106 and additional tokens generated from information stored by one or more additional data stores 110. The data integration system 114 may determine individual tokens generated from information stored by the health insurance claims data repository 106 that correspond to individual additional tokens generated from information stored by the one or more additional data repositories 110. By determining tokens generated using data stored by the health insurance claims data repository 106 that correspond to additional tokens generated using data stored by an additional data repository 110, the data integration system 114 may identify individuals having data that is stored in both the health insurance claims data repository 106 and in the additional data repository 110. In this way, the data integration system 114 may obtain data from the health insurance claims data repository 106 from a number of individuals and data from the additional data repository 110 from the same number of individuals and store the health insurance claims data and the additional data for the number of individuals in the integrated data repository 104. The health insurance claims data and the additional data stored by the integrated data repository 104 for the number of individuals may be accessible using respective identifiers of individuals.

In one or more further examples, the data integration system 114 may obtain tokens generated from information stored by the molecular data repository 108 and tokens generated from information stored by one or more additional data stores 110. The data integration system 114 may determine individual tokens generated from information stored by the molecular data repository 108 that correspond to individual additional tokens generated from information stored by the one or more additional data repositories 110. By determining tokens generated using data stored by the molecular data repository 108 that correspond to additional tokens generated using data stored by an additional data repository 110, the data integration system 114 may identify individuals having data that is stored in both the molecular data repository 108 and in the additional data repository 110. In this way, the data integration system 114 may obtain data from the molecular data repository 108 from a number of individuals and data from the additional data repository 110 from the same number of individuals and store the molecular data and the additional data for the number of individuals in the integrated data repository 104. The molecular data and the additional data stored by the integrated data repository 104 for the number of individuals may be accessible using respective identifiers of individuals.

The data stored by the integrated data repository 104 may be stored according to one or more regulatory frameworks that protect the privacy and ensure the security of medical records, health information, and insurance information of individuals. For example, data may be stored by the integrated data repository 104 in accordance with one or more governmental regulatory frameworks directed to protecting personal information, such as the Health Insurance Portability and Accountability Act (HIPAA) and/or the General Data Protection Regulation (GDPR). The integrated data repository 104 also stores data in an anonymized and de-identified manner to ensure protection of the privacy of individuals that have data stored by the integrated data repository 104. To further ensure the privacy of individuals that have data stored by the integrated data repository 104, the data integration system 114 may re-generate the integrated data repository 104 periodically. For example, the data integration system 114 may create the integrated data repository 104 once per quarter. In one or more additional examples, the data integration system 114 may generated the integrated data repository 104 on a monthly basis, on a weekly basis, or once every two weeks. By re-generating the integrated data repository 104 on a periodic basis and not simply refreshing the integrated data repository 104 when new data is available, the integrated data repository 104 enhances privacy protection with respect to data stored by the integrated data repository 104. That is, in situations where data repositories are refreshed simply with new data, it may be possible to more easily track individuals associated with data that has been newly added to a data repository because the number of new individuals added at a given time is typically smaller than an existing number of individuals that already have data stored by the data repository.

In various examples, data stored by the integrated data repository 104 may be accessed via a database management system. In addition, the integrated data repository 104 may store data according to one or more database models. In one or more examples, the integrated data repository 104 may store data according to one or more relational database technologies. For example, the integrated data repository 104 may store data according to a relational database model. In one or more additional examples, the integrated data repository 104 may store data according to an object-oriented database model. In one or more further examples, the integrated data repository 104 may store data according to an extensible markup language (XML) database model. In still additional examples, the integrated data repository 104 may store data according to a structured query language (SQL) database model. In still further examples, the integrated data repository may store data according to an image database model.

The data integration system 114 may generate the integrated data repository 104 by generating a number of data tables and creating links between the data tables. The links may indicate logical couplings between the data tables. The data integration system 114 may generate the data tables by extracting specified sets of data from the information obtained from the data repositories 106, 108, 110, 112 and storing the data in rows and columns of respective data tables. In various examples, the logical couplings between data tables may include at least one of a one-to-one link where a row of information in one data table corresponds to a row of information in another data table, a one-to-many link where a row of information in one data table corresponds to multiple rows of information in another data table, or a many-to-many link where multiple rows of information of one data table correspond to multiple rows of information in another data table.

The number of data tables may be arranged according to a data repository schema 116. In the illustrative example of FIG. 1, the database schema 114 includes a first data table 118, a second data table 120, a third data table 122, a fourth data table 124, and a fifth data table 124. Although the illustrative example of FIG. 1 includes five data tables, in additional implementations, the data repository schema 116 may include more data tables or fewer data tables. The data repository schema 116 may also include links between the data tables 118, 120, 122, 124, 128. The links between the data tables 118, 120, 122, 124, 126 may indicate that information retrieved from one of the data tables 118, 120, 122, 124, 126 results in additional information stored by one or more additional data tables 118, 120, 122, 124, 126 to be retrieved. Additionally, not all the data tables 118, 120, 122, 124, 126 may be linked to each of the other data tables 118, 120, 120, 122, 124, 126. In the illustrative example of FIG. 1, the first data table 118 is logically coupled to the second data table 118 by a first link 128 and the first data table 118 is logically coupled to the fourth data table 124 by a second link 130. In addition, the second data table 120 is logically coupled to the third data table 122 via a third link 132 and the fourth data table 124 is logically coupled to the fifth data table 126 via a fourth link 134. Further, the third data table 122 is logically coupled to the fifth data table 126 via a fifth link 136.

In various examples, as data tables are added to and/or removed from the data repository schema 116, additional links between data tables may be added to or removed from the data repository schema 116. In one or more illustrative examples, the integrated data repository 104 may store data tables according to the data repository schema 116 for at least a portion of the individuals for which the data integration system 114 obtained information from a combination of at least two of the health insurance claims data repository 106, the molecular data repository 108, the one or more additional data repositories 110, and the one or more reference information data repositories 112. As a result, the integrated data repository 104 may store respective instances of the data tables 118, 120, 122, 124, 126 according to the data repository schema 116 for thousands, tens of thousands, up to hundreds of thousands or more individuals.

The data integration and analysis system 102 may also include a data pipeline system 138. The data pipeline system 138 may include a number of algorithms, software code, scripts, macros, or other bundles of computer-executable instructions that process information stored by the integrated data repository 104 to generate additional datasets. The additional datasets may include information obtained from one or more of the data tables 118, 120, 122, 124, 126. The additional datasets may also include information that is derived from data obtained from one or more of the data tables 118, 120, 122, 124, 126. The components of the data pipeline system 138 implemented to generate a first additional dataset may be different from the components of the data pipeline system 138 used to generate a second additional dataset.

In one or more examples, the data pipeline system 138 may generate a dataset that indicates pharmacy treatments received by a number of individuals. In one or more illustrative examples, the data pipeline system 138 may analyze information stored in at least one of the data tables 118, 120, 122, 124, 126 to determine health insurance codes corresponding to pharmaceutical treatments received by a number of individuals. The data pipeline system 138 may analyze the health insurance codes corresponding to pharmaceutical treatments with respect to a library of data that indicates specified pharmaceutical treatments that correspond to one or more health insurance codes to determine names of pharmaceutical treatments that have been received by the individuals. In one or more additional examples, the data pipeline system 138 may analyze information stored by the integrated data repository 104 to determine medical procedures received by a number of individuals. To illustrate, the data pipeline system 138 may analyze information stored by one of the data tables 118, 120, 122, 124, 126 to determine treatments received by individuals via at least one of injection or intravenously. In one or more further examples, the data pipeline system 138 may analyze information stored by the integrated data repository 104 to determine episodes of care for individuals, lines of therapy received by individuals, progression of a biological condition, or time to next treatment. In various examples, the datasets generated by the data pipeline system 138 may be different for different biological conditions. For example, the data pipeline system 138 may generate a first number of datasets with respect to a first type of cancer, such as lung cancer, and a second number of datasets with respect to a second type of cancer, such as colorectal cancer.

The data pipeline system 138 may also determine one or more confidence levels to assign to information associated with individuals having data stored by the integrated data repository 104. The respective confidence levels may correspond to different measures of accuracy for information associated with individuals having data stored by the integrated data repository 104. The information associated with the respective confidence levels may correspond to one or more characteristics of individuals derived from data stored by the integrated data repository 104. Values of confidence levels for the one or more characteristics may be generated by the data pipeline system 138 in conjunction with generating one or more datasets from the integrated data repository 104. In one or more examples, a first confidence level may correspond to a first range of measures of accuracy, a second confidence level may correspond to a second range of measures of accuracy, and a third confidence level may correspond to a third range of measures of accuracy. In one or more additional examples, the second range of measures of accuracy may include values that are less values of the first range of measures of accuracy and the third range of measures of accuracy may include values that are less than values of the second range of measures of accuracy. In one or more illustrative examples, information corresponding to the first confidence level may be referred to as Gold standard information, information corresponding to the second confidence level may be referred to as Silver standard information, and information corresponding to the third confidence level may be referred to as Bronze standard information.

The data pipeline system 138 may determine values for the confidence levels of characteristics of individuals based on a number of factors. For example, a respective set of information may be used to determine characteristics of individuals. The data pipeline system 138 may determine the confidence levels of characteristics of individuals based on an amount of completeness of the respective set of information used to determine a characteristic for an individual. In situations where one or more pieces of information are missing from the set of information associated with a first number of individuals, the confidence levels for a characteristic may be lower than for a second number of individuals where information is not missing from the set of information. In one or more examples, an amount of missing information may be used by the data pipeline system 138 to determine confidence levels of characteristics of individuals. To illustrate, a greater amount of missing information used to determine a characteristic of an individual may cause confidence levels for the characteristic to be lower than in situations where the amount of missing information used to determine the characteristic is lower. Further, different types of information may correspond to various confidence levels for a characteristic. In one or more examples, the presence of a first piece of information used to determine a characteristic of an individual may result in confidence levels for the characteristic being higher than the presence of a second piece of information used to determine the characteristic.

In one or more illustrative examples, the data pipeline system 138 may determine a number of individuals included in a cohort with a primary diagnosis of lung cancer (or other biological condition). The data pipeline system 138 may determine confidence levels for respective individuals with respect to being classified as having a primary diagnosis of lung cancer. The data pipeline system 138 may use information from a number of columns included in the data tables 118, 120, 122 124, 126 to determine a confidence level for the inclusion of individuals within a lung cancer cohort. The number of columns may include health insurance codes related to diagnosis of biological conditions and/or treatments of biological conditions. Additionally, the number of columns may correspond to dates of diagnosis and/or treatment for biological conditions. The data pipeline system 138 may determine that a confidence level of an individual being characterized as being part of the lung cancer cohort is higher in scenarios where information is available for each of the number of columns or at least a threshold number of columns than in instances where information is available for less than a threshold number of columns. Further, the data pipeline system 138 may determine confidence levels for individuals included in a lung cancer cohort based on the type of information and availability of information associated with one or more columns. To illustrate, in situations where one or more diagnosis codes are present in relation to one or more periods of time for a group of individuals and one or more treatment codes are absent, the data pipeline system 138 may determine that the confidence level of including the group of individuals in the lung cancer cohort is greater than in situations where at least one of the diagnosis codes is absent and the treatment codes used to determine whether individuals are included in the lung cancer cohort are present.

The data integration and analysis system 102 may include a data analysis system 140. The data analysis system 140 may receive integrated data repository requests 142 from one or more computing devices, such as an example computing device 144. The one or more integrated data repository requests 142 may cause data to be retrieved from the integrated data repository 104. In various examples, the one or more integrated data repository requests 142 may cause data to be retrieved from one or more datasets generated by the data pipeline system 138. The integrated data repository requests 142 may specify the data to be retrieved from the integrated data repository 104 and/or the one or more datasets generated by the data pipeline system 138. In one or more additional examples, the integrated data repository requests 142 may include one or more prebuilt queries that correspond to computer-executable instructions that retrieve a specified set of data from the integrated data repository 104 and/or one or more datasets generated by the data pipeline system 138.

In response to one or more integrated data repository requests 142, the data analysis system 140 may analyze data retrieved from at least one of the integrated data repository 104 or one or more datasets generated by the data pipeline system 138 to generate data analysis results 146. The data analysis results 146 may be sent to one or more computing devices, such as example computing device 148. Although the illustrative example of FIG. 1 shows that the one or more integrated data repository requests 142 from one computing device 144 and the data analysis results 146 being sent to another computing device 148, in one or more additional implementations, the data analysis results 146 may be received by a same computing device that sent the one or more integrated data repository requests 142. The data analysis results 146 may be displayed by one or more user interfaces rendered by the computing device 144 or the computing device 148.

In one or more examples, the data analysis system 140 may implement at least one of one or more machine learning techniques or one or more statistical techniques to analyze data retrieved in response to one or more integrated data repository requests 142. In one or more illustrative examples, the data analysis system 140 may determine a rate of survival of individuals in which lung cancer is present in response to one or more treatments. In one or more additional illustrative examples, the data analysis system 140 may determine a rate of survival of individuals having one or more genomic region mutations in which lung cancer is present in response to one or more treatments. In various examples, the data analysis system 140 may generate the data analysis results 146 in situations where the data retrieved from at least one of the integrated data repository 104 or the one or more datasets generated by the data pipeline system 138 satisfies one or more criteria. For example, the data analysis system 140 may determine whether at least a portion of the data retrieved in response to one or more integrated data repository requests 142 satisfies a threshold confidence level. In situations where the confidence level for at least a portion of the date retrieved in response to one or more integrated data repository requests 142 is less than a threshold confidence level, the data analysis system 140 may refrain from generating at least a portion of data analysis results 146. In scenarios where the confidence level for at least a portion of the data retrieved in response to one or more integrated data repository requests 142 is at least a threshold confidence level, the data analysis system 140 may generate at least a portion of the data analysis results 146. In various examples, the threshold confidence level may be related to the type of data analysis results 146 being generated by the data analysis system 140.

In one or more illustrative examples, the data analysis system 140 may receive an integrated data repository request 142 to generate data analysis results 146 that indicate a rate of survival of one or more individuals. In these instances, the data analysis system 140 may determine whether the data stored by the integrated data repository 104 and/or by one or more datasets generated by the data pipeline system 138 satisfies a threshold confidence level, such as a Gold standard confidence level. In one or more additional examples, the data analysis system 140 may receive an integrated data repository request 142 to generate data analysis results 146 that indicate a treatment received by one or more individuals. In these implementations, the data analysis system 140 may determine whether the data stored by the integrated data repository 104 and/or by one or more datasets generated by the data pipeline system 138 satisfies a lower threshold confidence level, such as a Bronze standard confidence level.

The data integration and analysis system 102 can also include a treatment reference table system 150. Although the treatment reference table system 150 is shown in the illustrative example of FIG. 1 as being separate from the data integration system 114 and the data pipeline system 138, at least a portion of the operations performed by the treatment reference table system 150 can be performed by at least one of the data integration system 114 or the data pipeline system 138. The treatment reference table system 150 can analyze information obtained from the health insurance claims data repository 106 and at least one reference information data repository 112 to generate a treatment reference table 152. Data included in the treatment reference table 152 can be at least one of accessed or provided to the data analysis system 140 in response to one or more integrated data repository requests 142 to generate the data analysis results 146.

In one or more examples, the treatment reference table system 150 can analyze health insurance claims data to determine insurance code identifiers that correspond to treatments. The treatment reference table system 150 can then extract insurance code identifiers from the health insurance claims data that corresponds to treatments provided to individuals having data stored by the integrated data repository 104. The insurance code identifiers that correspond to treatments can be analyzed with respect to one or more criteria related to the format of the insurance code identifiers. Additionally, the insurance code identifiers can be used in generating one or more API requests to obtain information from a reference information data repository 112 that is related to insurance code identifiers and treatments corresponding to the insurance code identifiers. In one or more illustrative examples, the treatment reference table system 150 can determine that an insurance code identifier that corresponds to a treatment is formatted according to an NDC-9 format or an NDC-10 format. The treatment reference table system 150 can generate an API request that is sent to a reference information data repository 112 and a response can be returned including information from the reference information data repository 112 that has an additional insurance code identifier with an NDC-11 format that corresponds to the initial insurance code identifier that had the NDC-9 format or the NDC-10 format.

The treatment reference table system 150 can generate additional API requests using valid insurance code identifiers that satisfy the one or more formatting criteria. The additional API requests can be sent to a reference information data repository 112 to obtain additional information related to the valid insurance code identifiers. For example, the treatment reference table system 150 can obtain additional information about an insurance code identifier that includes at least one of a name of a treatment that corresponds to the insurance code identifier, one or more ingredients of a treatment that corresponds to the insurance code identifier, at least one class of treatments corresponding to the insurance code identifier, at least one source related to the class information, an additional identifier of the insurance code identifier within the reference information data repository 112, or a term type related to the treatment.

The treatment reference table system 150 can generate the treatment reference table 152 using the information obtained in response to API requests sent to one or more reference information data repositories 112. In one or more additional examples, the treatment reference table system 150 can generate the treatment reference table 152 using information obtained from the health insurance claims data repository 106. For example, the treatment reference table system 150 can create a row of the treatment reference table 152 for a unique insurance code identifier included in data obtained from the health insurance claims data repository 106. A unique insurance code identifier can include an insurance code identifier obtained from the health insurance claims data repository 106 that does not include a same set of alphanumeric characters or other symbols arranged in a same order as any other insurance code identifier obtained from the health insurance claims data repository 106.

The treatment reference table 152 can also include a number of columns that correspond to individual rows of the treatment reference table 152. In one or more examples, the treatment reference table 152 can include a column that indicates an insurance code identifier obtained from the health insurance claims data repository 106 and an additional identifier that corresponds to the insurance code identifier that is obtained from a reference information data repository 112. In addition, the treatment reference table 152 can include a column that indicates a name of a treatment that corresponds to an insurance code identifier. Further, the treatment reference table 152 can include a column that indicates a class of a treatment and a column that indicates a source of the class. In various examples, a source of a class can correspond to a classification scheme used to organize and/or characterize classes of treatments. In one or more additional examples, the treatment reference table 152 can include a column that includes a comment that is generated by the treatment reference table system 150. The comment can be related to the treatment that corresponds to a given row and/or information about the treatment. In one or more examples, one or more columns can include a null value.

In one or more implementations, the reference information data repository 112 storing the information requested with respect to the insurance code identifiers and treatments can be external to an entity that is associated with the data integration and analysis system 102. In one or more additional examples, the reference information data repository 112 storing the information requested with respect to insurance code identifiers and treatments can be an internal data repository that is controlled and maintained by an entity that also controls, implements, and/or maintains the data integration and analysis system 102. To illustrate, the internal reference information data repository 112 storing the information related to insurance code identifiers and treatments can store copies of information obtained from an external reference information data repository 112. In various examples, the internal reference information data repository 112 can be updated periodically using a number of API requests to the external reference information data repository 112 that store the insurance code identifier and treatment information.

In one or more illustrative examples, the data integration and analysis system 102 can obtain an integrated data repository request 142 that includes at least one of a respective name of one or more treatments, an ingredient included in one or more treatments, or a class of one or more treatments. The use of a name, ingredient, or class of a treatment is more commonly known than insurance code identifiers that can be related to treatments and, thus, enables queries of the integrated data repository 104 to be generated more easily than in situations where insurance code identifiers are used that often change and/or are not readily available. After receiving an integrated data repository request 142 that includes at least one of a respective name, ingredient, or class of one or more treatments, the data analysis system 140 can use the treatment reference table 152 to determine one or more insurance code identifiers that correspond to the information included in the integrated data repository request 142. The data analysis system 140 can then query the integrated data repository 104 to determine individuals that correspond to the treatment. In this way, a cohort of individuals that corresponds to a treatment can be determined based on a query to the data analysis system 150 that includes at least one of a respective name of the treatment, an ingredient included in the treatment, or a class of the treatment. One or more additional analyses of information related to the individuals included in the cohort can then be performed. For example, genetic information of individuals included in the cohort can be analyzed. Additionally, dosage information and/or frequency with which the treatment is received with respect to individuals included in the cohort can be analyzed. Further, diagnosis information of individuals included in the cohort can be analyzed. Outcomes of at least a portion of the analysis performed by the data analysis system 140 using the treatment reference table 152 can be included in the data analysis results 146.

In one or more additional illustrative examples, the data analysis system 140 may receive a request to analyze information that corresponds to a cohort of patients. One or more genomic mutations may be present in the cohort of individuals. In addition, the patients included in the cohort may have received treatment for a given biological condition. In response to the request, the data analysis system 140 may analyze information stored by the integrated data repository 104 to generate data analysis results 146 that include one or more quantitative measures corresponding to patients included in the cohort. To illustrate, the data analysis system 140 may determine real world survival metrics for patients included in the cohort. In various examples, the data analysis system 140 may analyze information related to a cohort of patients to determine a survival probability over a period of time for patients included in the cohort. In one or more illustrative examples, the data analysis system 140 may analyze information related to one or more cohorts of patients to determine real-world overall survival metrics for the patients included in the cohort. In one or more further illustrative examples, the data analysis system 140 may analyze information related to the cohort to determine time-to-next-treatment metrics and/or time to discontinuation metrics for patients included in the cohort.

In various examples, the data analysis system 140 may analyze information that corresponds to patients included in the cohort to determine an amount of progression of the biological condition within at least a subset of the patients included in the cohort. In one or more examples, the data analysis system 140 may determine an amount of progression for a cohort of patients receiving one or more pharmaceutical substances as part of a line of therapy. In one or more illustrative examples, the data analysis system 140 may analyze at least one of time-to-next-treatment metrics or time to discontinuation metrics for a cohort of patients to determine an amount of progression of the biological condition for patients of the cohort. In these instances, the data analysis system 140 may query the integrated data repository 1104 to determine genomic data of patients included in the cohort and identify patients of the cohort having one or more specified genomic mutations. The data analysis system 140 may then analyze time-to-next-treatment metrics, time to discontinuation metrics, and/or real-world overall survival metrics of patients included in the cohort having the one or more genomic mutations to determine progression of a biological condition for patients included in the cohort and that received the treatment for the biological condition.

In one or more further examples, the data analysis system 140 may analyze information about the cohort to determine a level of resistance developed by one or more patients included in the cohort receiving one or more treatments for a biological condition. In various examples, the data analysis system 140 may analyze at least one of time-to-next-treatment metrics, time to discontinuation metrics, or real-world survival metrics to determine a level of resistance developed by patients of the cohort that received treatment for the biological condition. In at least some examples, the data analysis system 140 may also determine a level of resistance with respect to one or more treatments for patients in the cohort having one or more genomic mutations. In at least some examples, the level of resistance may be greater in situations where a time-to-next-treatment or a real world survival rate have lower values and the level of resistance may be lower in situations where values of time-to-next-treatment or real-world survival rate are relatively higher.

In at least some examples, the data analysis system 140 may analyze lines of therapy information to determine a recommendation for one or more treatments to administer to a patient diagnosed with a biological condition. In one or more examples, the data analysis system 140 may analyze information about cohorts of patients to determine one or more characteristics of patients of the cohort that received one or more lines of therapy in which a level of resistance is relatively low and/or an amount of progression is relatively low. The data analysis system 140 may then analyze characteristics of one or more additional patients of the cohort diagnosed with the biological condition to determine whether to recommend the one or more lines of therapy as treatment to the one or more additional patients. At least a portion of the one or more additional patients of the cohort may have already received treatment for the biological condition. In one or more additional examples, at least a portion of the one or more additional patients of the cohort may not have received treatment for the biological condition associated with the cohort. In various examples, the data analysis system 140 may also analyze information of patients included in a given cohort to determine an effectiveness of a line of therapy for the patients included in the cohort. The effectiveness of the line of therapy may correspond to a probability of the line of therapy at least one of reducing the effects of or eliminating the biological condition with respect the patients of the cohort.

In various examples, an amount of progression of the biological condition, an effectiveness of a line of therapy to treat the biological condition, the probability of developing resistance to a line of treatment, or a combination thereof, may be determined by the data analysis system 140 using at least one of one or more statistical techniques or one or more machine learning techniques. To illustrate, the data analysis system 140 may implement at least one of Cox proportional hazards models, chi-squared tests, log-rank tests, or Kaplan-Meier methods to determine at least one of an amount of progression of the biological condition, an effectiveness of a line of therapy to treat the biological condition, or the probability of developing resistance to a line of treatment. In one or more additional examples, the data analysis system 140 may implement one or more neural networks, one or more convolutional neural networks, or one or more residual neural networks to determine at least one of an amount of progression of the biological condition, an effectiveness of a line of therapy to treat the biological condition, or the probability of developing resistance to a line of treatment.

One or more therapeutically effective amounts of one or more treatments may be administered to one or more patients included in a cohort based on an amount of progression of the biological condition, a level of effectiveness of one or more lines of therapy, or a level of resistance determined with respect to the one or more patients. The therapeutically effective amounts of the one or more treatments can correspond to a new line of therapy or an additional line of therapy for the one or more patients. In various examples, the therapeutically effective amounts of the one or more treatments can be provided to replace ineffective treatments previously provided to the one or more patients, such as due to the one or more patients developed a threshold level of resistance to one or more previous treatments provided to the one or more patients.

In one or more examples, the data integration and analysis system 102 can identify treatments to administer to patients having a given disease, disorder, or biological condition. Essentially any cancer therapy (e.g., surgical therapy, radiation therapy, chemotherapy, and/or the like) is included as part of these treatment. In various examples, the treatment administered to a patient can include at least one chemotherapy drug. In at least some examples, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In one or more additional examples, the chemotherapy administered to a subject can comprise FOLFOX or FOLFIRI. The treatments can also include various poly adenosine diphosphate-ribose polymerase (PARP) inhibitors, such as rucaparib and niraparib, in addition to kinase inhibitors, such as Larotrectinib, binimetinib, encorafenib and tofacitinib. In various examples, the one or more treatments can be administered to treat one or more forms of cancer, such as entrectinib, dacomitinib, and topotecan to treat lung cancer; trifluridine/tipracil, and irinotecan to treat colon cancer; apalutamide, degarelix, abiraterone, and enzalutamide to treat prostate cancer; and tucatinib, talazoparib, and olaparib to treat breast cancer.

In one or more further examples, the one or more treatments can include at least one immunotherapy (or an immunotherapeutic agent). Immunotherapy refers generally to methods of enhancing an immune response against a given cancer type. In at least some implementations, immunotherapy refers to methods of enhancing a T cell response against a tumor or cancer. In various examples, the immunotherapy or immunotherapeutic agents target an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In various examples, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In one or more examples, the inhibitory immune checkpoint molecule is CTLA4 or PD-1. In one or more additional examples, the inhibitory immune checkpoint molecule is a ligand for PD-1, such as PD-L1 or PD-L2. In at least some examples, the inhibitory immune checkpoint molecule is a ligand for CTLA4, such as CD80 or CD86. In still other examples, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in one or more implementations, the immunotherapy or immunotherapeutic agent is an antagonist of an inhibitory immune checkpoint molecule. In various examples, the inhibitory immune checkpoint molecule is PD-1. In one or more examples, the inhibitory immune checkpoint molecule is PD-L1. In one or more additional examples, the antagonist of the inhibitory immune checkpoint molecule is an antibody (e.g., a monoclonal antibody). In one or more further examples, the antibody or monoclonal antibody is an anti-CTLA4, anti-PD-1, anti-PD-L1, or anti-PD-L2 antibody. In still other examples, the antibody is a monoclonal anti-PD-1 antibody. In at least some examples, the antibody is a monoclonal anti-PD-L1 antibody. In various examples, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In one or more instances, the anti-PD-1 antibody is one or more of pembrolizumab (Keytruda®) or nivolumab (Opdivo®). In various scenarios, the anti-CTLA4 antibody is ipilimumab (Yervoy®). In at least some implementations, the anti-PD-L1 antibody is one or more of atezolizumab (Tecentriq®), avelumab (Bavencio®), or durvalumab (Imfinzi®).

In one or more examples, the immunotherapy or immunotherapeutic agent is an antagonist (e.g. antibody) against CD80, CD86, LAG3, KIR, TIM3, GAL9, or A2aR. In one or more additional examples, the antagonist is a soluble version of the inhibitory immune checkpoint molecule, such as a soluble fusion protein comprising the extracellular domain of the inhibitory immune checkpoint molecule and an Fc domain of an antibody. In one or more further, the soluble fusion protein comprises the extracellular domain of CTLA4, PD-1, PD-L1, or PD-L2. In at least some examples, the soluble fusion protein comprises the extracellular domain of CD80, CD86, LAG3, KIR, TIM3, GAL9, or A2aR. In various implementations, the soluble fusion protein comprises the extracellular domain of PD-L2 or LAG3.

In one or more implementations, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In at least some examples, the immune checkpoint molecule is a co-stimulatory molecule selected from CD28, inducible T cell co-stimulator (ICOS), CD137, OX40, or CD27. In various examples, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in one or more examples, the immunotherapy or immunotherapeutic agent is an agonist of a co-stimulatory checkpoint molecule. In one or more additional examples, the agonist of the co-stimulatory checkpoint molecule is an agonist antibody and preferably is a monoclonal antibody. In one or more further implementations, the agonist antibody or monoclonal antibody is an anti-CD28 antibody. In at least some examples, the agonist antibody or monoclonal antibody is an anti-ICOS, anti-CD137, anti-OX40, or anti-CD27 antibody. In various examples, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In one or more implementations, the customized therapies described herein are typically administered parenterally (e.g., intravenously or subcutaneously). Pharmaceutical compositions containing the immunotherapeutic agent are typically administered intravenously. Certain therapeutic agents are administered orally. Customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In one or more further illustrative examples, the data integration and analysis system 102 can analyze data stored by one or more of the data repositories 106, 108, 110, 112 to generate an additional data table that indicates information about one or more patients. In at least some examples, the one or more patients may have received one or more treatments for one or more biological conditions. In one or more illustrative examples, the additional data table can include information about a cohort of patients in which a biological condition is present and that have received one or more specified treatments in relation to the biological condition. In one or more additional illustrative examples, the additional data table can include information that corresponds to an additional cohort of individuals in which the biological condition is not present. In various examples, the individuals included in the additional cohort can be labeled as healthy individuals.

The additional data table can include a number of columns with individual columns corresponding to individual patients. The additional data table can also include a number of rows with individual rows corresponding to a feature of individual patients. The features can include numerical indicators that correspond to genomic mutations, biometric data, results of analytical tests, diagnostic imaging procedures, other diagnostic test results, physical characteristics of patients, personal information of patients, quantitative bioinformatics information, one or more combinations thereof, and the like. In this way, the additional data table can include a high-dimensional data matrix of column vectors representative of a number of features of individual patients. In various examples, the information stored by the additional data table can be analyzed to determine a biological state of each individual. The biological state can be determined according to a number of different criteria. To illustrate, the biological state of individual patients can correspond to a level of overall health where data related to individuals in which one or more specified biological conditions are not present is used to determine a baseline level of health and data of patients in which the one or more specified biological conditions are present are measured against the baseline level of health. The biological state of individual patients can also be determined in relation to at least one of the presence of one or more biological conditions, a level in which the one or more biological conditions are present, or the absence of the one or more specified biological conditions. In at least some examples, the biological state of patients can vary according to at least one of age, diet, ethnic background, disease status, lifestyle choices, location, or environment.

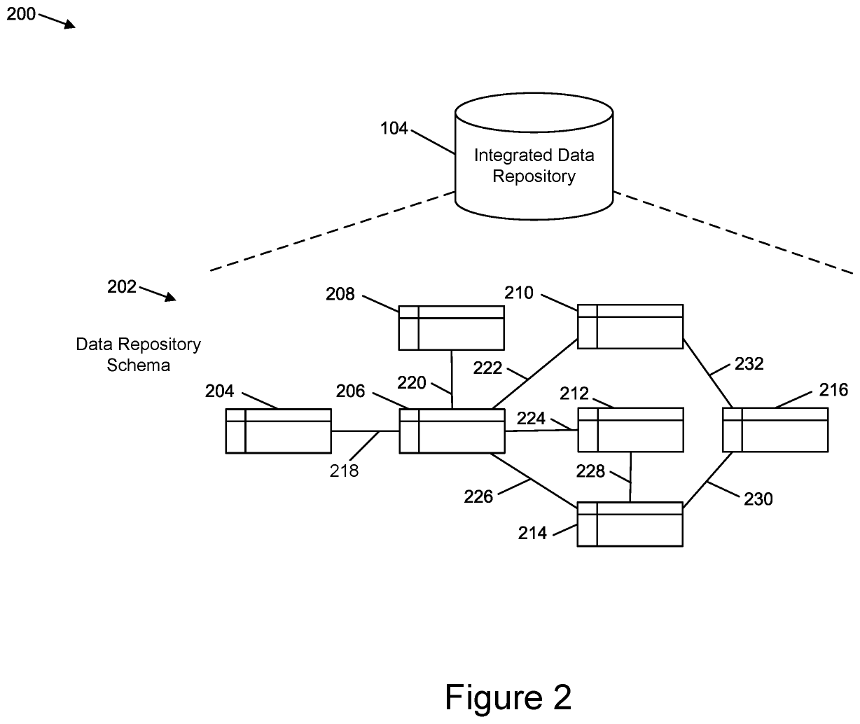

FIG. 2 illustrates an example framework 200 corresponding to an arrangement of data tables in an integrated data repository, according to one or more implementations. In the illustrative example of FIG. 2, the framework 200 includes a database schema 202 that includes a first data table 204, a second data table 206, a third data table 208, a fourth data table 210, a fifth data table 212, a sixth data table 214, and a seventh data table 216. Although the illustrative example of FIG. 2 includes seven data tables, in additional implementations, the data repository schema 202 may include more data tables or fewer data tables. The data repository schema 202 may also include links between the data tables 204, 206, 208, 210, 212, 214, 216. The links between the data tables 204, 206, 208, 210, 212, 214, 216 may indicate that information retrieved from one of the data tables 204, 206, 208, 210, 212, 214, 216 results in additional information stored by one or more additional data tables 204, 206, 208, 210, 212, 214, 216 to be retrieved. Additionally, not all the data tables 204, 206, 208, 210, 212, 214, 216 may be linked to each of the other data tables 204, 206, 208, 210, 212, 214, 216. In the illustrative example of FIG. 2, the first data table 204 is logically coupled to the second data table 206 by a first link 218 and the third data table 208 is logically coupled to the second data table 206 by a second link 220. The second data table 206 is also logically coupled to the fourth data table 210 by a third link 222, the second data table 206 is logically coupled to the fifth data table 212 by a fourth link 224, and the second data table 206 is logically coupled to the sixth data table 214 by a fifth link 226. In addition, fifth data table 212 is logically coupled to the sixth data table 214 by a sixth link 228 and the sixth data table 214 is logically coupled to the seventh data table 216 by a seventh link 230. Further, the seventh data table 216 is logically coupled to the fourth data table 210 by an eighth link 232. In various examples, as data tables are added to and/or removed from the data repository schema 202, additional links between data tables may be added to or removed from the data repository schema 202. In one or more illustrative examples, the integrated data repository 104 may store data tables according to the data repository schema 202 for at least a portion of the individuals for which the data integration system 114 obtained information from a combination of at least two of the health insurance claims data repository 106, the molecular data repository 108, and the one or more additional data repositories 110. As a result, the integrated data repository 104 may store respective instances of the data tables 204, 206, 208, 210, 212, 214, 216 according to the data repository schema 204 for thousands, tens of thousands, up to hundreds of thousands or more individuals.

In one or more examples, the first data table 204 may store data corresponding to genomics and genomics testing for individuals. For example, the first data table 204 may include columns that include information corresponding to a panel used to generate genomics data, mutations of genomic regions, types of mutations, copy numbers of genomic regions, coverage data indicating numbers of nucleic acid molecules identified in a sample having one or more mutations, testing dates, and patient information. The first data table 204 may also include one or more columns that include health insurance data codes that may correspond to one or more diagnosis codes. Additionally, the information in first data table 204 may include at least one identifier for an individual that is associated with an instance of the first data table 204.

The second data table 206 may store data related to one or more patient visits by individuals to one or more healthcare providers. The third data table 208 may store information corresponding to respective services provided to individuals with respect to one or more patient visits to one or more healthcare providers indicated by the second data table 206. To illustrate, an individual may visit a healthcare provider and multiple services may be performed with respect to the individual at the visit. A second data table 206 may include columns indicating information for each of the multiple services performed during the patient visit. Multiple third data tables 208 may be generated with respect to the patient visit that include columns indicating information on a more granular level for a respective service provided during the patient visit than the information stored by the second data table 206 related to the patient visit. For example, the second data table 206 may include multiple columns indicating a health insurance code for different services provided to an individual during a patient visit and a third data table 208 related to one of the services may include multiple columns for additional health insurance codes that correspond to additional information related to the respective services. The second data table 206 and the third data table(s) 208 for a patient visit may indicate one or more dates of service corresponding to the patient visit.

The fourth data table 210 may include columns that indicate information about individuals for which information is stored by the integrated data repository 104. For example, the fourth data table 210 may include columns that indicate information related to at least one of a location of an individual, a gender of an individual, a date of birth of an individual, a date of death of an individual (if applicable), or one or more keys associated with the individual. In one or more examples, the fourth data table 210 may include one or more columns related to whether erroneous data has been identified for an individual. In various examples, a single fourth data table 210 may be generated for respective individuals. Thus, the data repository schema 202 may include multiple instances of the fourth data table 210, such as thousands, tens of thousands, up to hundreds of thousands or more.

The fifth data table 212 may include columns that indicate information related to a health insurance company or governmental entity that made payment for one or more services provided to respective individuals. For example, the fifth data table 212 may include one or more payer identifiers. The sixth data table 214 may include columns that include information corresponding to health insurance coverage information for respective individuals. In one or more examples, the sixth data table 214 may include columns indicating the presence of medical coverage for an individual, the presence of pharmacy coverage for an individual, and a type of health insurance plan related to the individual, such as health maintenance organization (HMO), preferred provider organization (PPO), and the like.

The seventh data table 216 may include columns that indicate information related to pharmaceutical treatments obtained by a respective individual. In one or more examples, the seventh data table 216 may include one or more columns indicating health insurance codes corresponding to pharmaceutical treatments that are available via a pharmacy. The health insurance codes may correspond to individual pharmaceutical treatments. Additionally, the health insurance codes may indicate a diagnosis of a biological condition with respect to an individual. The seventh data table 216 may also include additional information, such as at least one of dosage amounts, number of days' supply, quantity dispensed, number of refills authorized, dates of service, or information related to the individual receiving the pharmaceutical treatment.

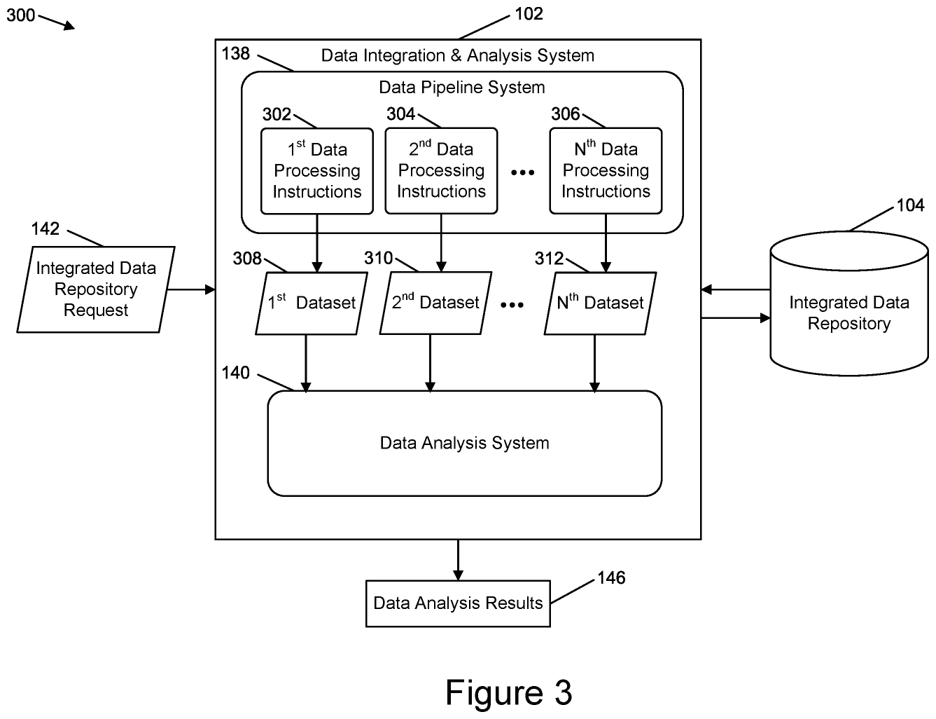

FIG. 3 illustrates an architecture 300 to generate one or more datasets from information retrieved from a data repository that integrates health related data from a number of sources, according to one or more implementations. The architecture 300 may include the data integration and analysis system 102 and the integrated data repository 104. Additionally, the data integration and analysis system 102 may include at least the data pipeline system 138 and the data analysis system 140. The data pipeline system 138 may include a number of sets of data processing instructions that are executable to generate respective datasets that may be analyzed by the data analysis system 140 in response to an integrated data repository request 142 to generate data analysis results 146.

The data pipeline system 138 may include first data processing instructions 302, second data processing instructions 304, up to Nth data processing instructions 306. The data processing instructions 302, 304, 306 may be executable by one or more processing units to perform a number of operations to generate respective datasets using information obtained from the integrated data repository 104. In one or more illustrative examples, the data processing instructions 302, 304, 306 may include at least one of software code, scripts, API calls, macros, and so forth. The first data processing instructions 302 may be executable to generate a first dataset 308. In addition, the second data processing instructions 304 may be executable to generate a second dataset 310. Further, the Nth data processing instructions 306 may be executable to generate an Nth dataset 312. In various examples, after the data integration and analysis system 102 generates the integrated data repository 104, the data pipeline system 138 may cause the data processing instructions 302, 304, 306 to be executed to generate the datasets 308, 310, 312. In one or more examples, the datasets 308, 310, 312 may be stored by the integrated data repository 104 or by an additional data repository that is accessible to the data integration and analysis system 102. At least a portion of the data processing instructions 302, 304, 306 may analyze health insurance codes to generate at least a portion of the datasets 308, 310, 312. Additionally, at least a portion of the data processing instructions 302, 304, 306 may analyze genomics data to generate at least a portion of the datasets 308, 310, 312.

In one or more examples, the first data processing instructions 302 may be executable to retrieve data from one or more first data tables stored by the integrated data repository 104. The first data processing instructions 302 may also be executable to retrieve data from one or more specified columns of the one or more first data tables. In various examples, the first data processing instructions 302 may be executable to identify individuals that have a health insurance code stored in one or more column and row combinations that correspond to one or more diagnosis codes. The first data processing instructions 302 may then be executable to analyze the one or more diagnosis codes to determine a biological condition for which the individuals have been diagnosed. In one or more illustrative examples, the first data processing instructions 302 may be executable to analyze the one or more diagnosis codes with respect to a library of diagnosis codes that indicates one or more biological conditions that correspond to respective diagnosis codes. The library of diagnosis codes may include hundreds up to thousands of diagnosis codes. The first data processing instructions 302 may also be executable to determine individuals diagnosed with a biological condition by analyzing timing information of the individuals, such as dates of treatment, dates of diagnosis, dates of death, one or more combinations thereof, and the like.

The second data processing instructions 304 may be executable to retrieve data from one or more second data tables stored by the integrated data repository 104. The second data processing instructions 304 may also be executable to retrieve data from one or more specified columns of the one or more second data tables. In various examples, the second data processing instructions 304 may be executable to identify individuals that have a health insurance code stored in one or more column and row combinations that correspond to one or more treatment codes. The one or more treatment codes may correspond to treatments obtained from a pharmacy. In one or more additional examples, the one or more treatment codes may correspond to treatments received by a medical procedure, such as an injection or intravenously. The second data processing instructions 304 may be executable to determine one or more treatments that correspond to the respective health insurance codes included in the one or more second data tables by analyzing the health insurance code in relation to a predetermined set of information. The predetermined set of information may include a data library that indicates one or more treatments that correspond to one out of hundreds up to thousands of health insurance codes. The second data processing instructions 304 may generate the second dataset 310 to indicate respective treatments received by a group of individuals. In one or more illustrative examples, the group of individuals may correspond to the individuals included in the first dataset 308. The second dataset 310 may be arranged in rows and columns with one or more rows corresponding to a single individual and one or more columns indicating the treatments received by the respective individual.

The Nth processing instructions 306 (where N may be any positive integer) may be executable to generate the Nth dataset 312 by combining information from a number of previously generated datasets, such as the first dataset 308 and the second dataset 310. In addition, the Nth processing instructions 306 may be executable to generate the Nth dataset 312 to retrieve additional information from one or more additional columns of the integrated data repository 104 and incorporate the additional information from the integrated data repository 104 with information obtained from the first dataset 308 and the second dataset 310. For example, the Nth processing instructions 306 may be executable to identify individuals included in the first dataset 308 that are diagnosed with a biological condition and analyze specified columns of one or more additional data tables of the integrated data repository 104 to determine dates of the treatments indicated in the second dataset 210 that correspond to the individuals included in the first dataset 308. In one or more further examples, the Nth processing instructions 306 may be executable to analyze columns of one or more additional data tables of the integrated data repository 104 to determine dosages of treatments indicated in the second dataset 310 received by the individuals included in the first dataset 308. In this way, the Nth processing instructions 306 may be executable to generate an episodes of care dataset based on information included in a cohort dataset and a treatments dataset.

In one or more illustrative examples, in response to receiving an integrated data repository request 142, the data analysis system 140 may determine one or more datasets that correspond to the features of the query related to the integrated data repository request 142. For example, the data analysis system 140 may determine that information included in the first dataset 308 and the second dataset 310 is applicable to responding to the integrated data repository request 142. In these scenarios, the data analysis system 140 may analyze at least a portion of the data included in the first dataset 308 and the second dataset 310 to generate the data analysis results 146. In one or more additional examples, the data analysis system 140 may determine different datasets to respond to different queries included in the integrated data repository request 142 in order to generate the data analysis results 146.

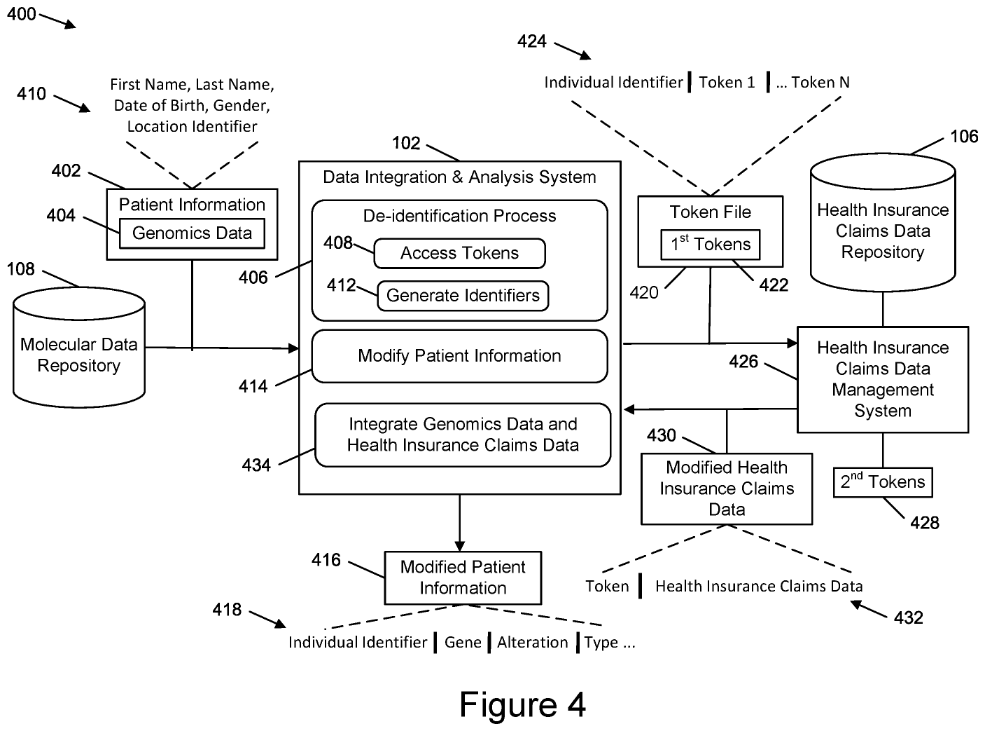

FIG. 4 illustrates an architecture 400 to generate an integrated data repository that includes de-identified health insurance claims data and de-identified genomics data it, according to one or more implementations. The architecture 400 may include the data integration and analysis system 102, the health insurance claims data repository 106, and the molecular data repository 108. The data integration and analysis system 102 may obtain patient information 402 from the molecular data repository 108. The patient information 402 may include genomics data 404 for individuals having data stored by the molecular data repository 108. The genomics data 404 may indicate results of one or more nucleic acid sequencing operations that analyze sequences of nucleic acid molecules included in a sample obtained from the individuals with respect to one or more target genomic regions. In one or more examples, the sample may be obtained from tissue of one or more individuals. In one or more additional examples, the sample may be obtained from fluid of one or more individuals, such as blood or plasma. The one or more target genomic regions may correspond to genomic regions that correspond to the presence of one or more biological conditions. For example, the target regions may correspond to genomic regions of a reference genome having mutations that are present in individuals in which a biological condition is present. In one or more illustrative examples, the target regions may correspond to genomic regions of a reference human genome in which one or more mutations are present in individuals in which one or more forms of cancer are present. The patient information 402 may also include information indicating personal information about individuals with data stored by the molecular data repository 108 and information corresponding to the testing and analysis performed on samples provided by individuals.

The data integration and analysis system 102 may perform a de-identification process 406 that anonymizes personal information obtained from the molecular data repository 108. The data integration and analysis system 102 may implement one or more computational techniques as part of the de-identification process to anonymize data related to individuals stored by the molecular data repository 108 such that the de-identified data protects the privacy of the individuals and is in compliance with one or more privacy regulation frameworks. The de-identification process 406 may include, at 408, accessing tokens. In various examples, the tokens may comprise an alphanumeric string of characters. In one or more examples, the tokens may be generated by the data integration and analysis system 102. In one or more additional examples the tokens may be generated by a third-party and obtained by the data integration and analysis system 102.

The tokens may be generated using one or more hash functions in relation to a subset 410 of the patient information 402. To illustrate, for individuals that have information stored by the molecular data repository 108, the tokens may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals. The de-identification process 406 may also include, at 412, generating identifiers for individuals that have data stored by the molecular data repository 108. The identifiers may be generated by the data integration and analysis system 102 using one or more hash functions that are different from the one or more hash functions used to generate the tokens. In one or more illustrative examples, the data integration and analysis system 102 may generate an intermediate version of respective identifiers using one or more hash function and then apply one or more salting techniques to the intermediate versions of the identifiers to generate final versions of the identifiers. In various examples, the data integration and analysis system 102 may generate the identifiers at 412 using at least a portion of the information for respective individuals stored by the molecular data repository 108. In one or more illustrative examples, the identifiers may be generated based on a patient identifier included in the patient information 402. The identifiers generated by the data integration and analysis system 102 may be unique for respective individuals having data stored by the molecular data repository 108.

At operation 414, the data integration and analysis system 102 may generate modified patient information 416 based on the identifiers. The modified patient information 416 may include genomics data 404 related to individuals associated with the molecular data repository 108 and the identifiers of the respective individuals. The modified patient information 416 may have a data structure 418. The data structure 418 may include a column that includes respective identifiers of individuals associated with the molecular data repository 108 and a number of columns that include genomics data 404 related to the individuals, such as identifiers of one or more genes, alterations to the one or more genes, type of alteration to the genes, and so forth.

The data integration and analysis system 102 may generate a token file 420. The token file 420 may include first tokens 422 accessed at operation 408 for respective individuals having data stored by the molecular data repository 108. The token file 420 may have a data structure 424 that includes a number of columns that include information for respective individuals. The data structure 424 may include a column indicating respective identifiers generated by the data integration and analysis system 102 and columns indicating one or more first tokens 422 associated with the respective identifiers. The data integration and analysis system 102 may send the token file 420 to a health insurance claims data management system 426 that is coupled to the health insurance claims data repository 106. The health insurance claims data management system 426 may analyze the first tokens 422 with respect to corresponding second tokens 428. The second tokens 428 may be accessed by or generated by the health insurance claims data management system 426. The second tokens 428 may be generated using a same or similar subset of information for individuals having data stored in the health insurance claims data repository 106 as the subset 410 of the patient information 402. For example, the second tokens 428 may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals.

In various examples, the health insurance claims data management system 426 may retrieve health insurance claims data from the health insurance claims data repository 106 for individuals associated with respective second tokens 428 that match corresponding first tokens 422. A first token 422 may match a second token 428 when the data of the first token 422 has at least a threshold amount of similarity with respect to the data of the second token 428. In one or more examples, a first token 422 may match a second token 428 when the data of the first token 422 is the same as the data of the second token 428.

In response to identifying health insurance claims data for individuals having respective second tokens 428 that correspond to a respective first token 422, the health insurance claims data management system 426 may generate modified health insurance claims data 430. The health insurance claims data management system 426 may send the modified health insurance claims data 430 to the data integration and analysis system 102. In one or more examples, the modified health insurance claims data 430 may be formatted according to a data structure 432. The data structure 432 may include a column that includes a subset of the second tokens 428 that correspond to the first tokens 422 and a number of columns that include the health insurance claims data.

At operation 434, the data integration and analysis system 102 may integrate genomics data and health insurance claims data of individuals that are common to both the molecular data repository 108 and the health insurance claims data repository 106. The data integration and analysis system 102 may determine individuals that are common to both the molecular data repository 108 and the health insurance claims data repository 106 by determining genomics data and health insurance claims data corresponding to common tokens. The data integration and analysis system 102 may determine that a first token 422 related to a portion of the genomics data 404 corresponds to a second token 428 related to a portion of the health insurance claims data by determining a measure of similarity between the first token 422 and the second token 428. In scenarios where the first token 422 has at least a threshold amount of similarity with respect to the second token 428, the data integration and analysis system 102 may store the corresponding portion of the genomics data 404 and the corresponding portion of the health insurance claims data in relation to the identifier of the individual in an integrated data repository, such as the integrated data repository 104 of FIG. 1, FIG. 2, and FIG. 3.

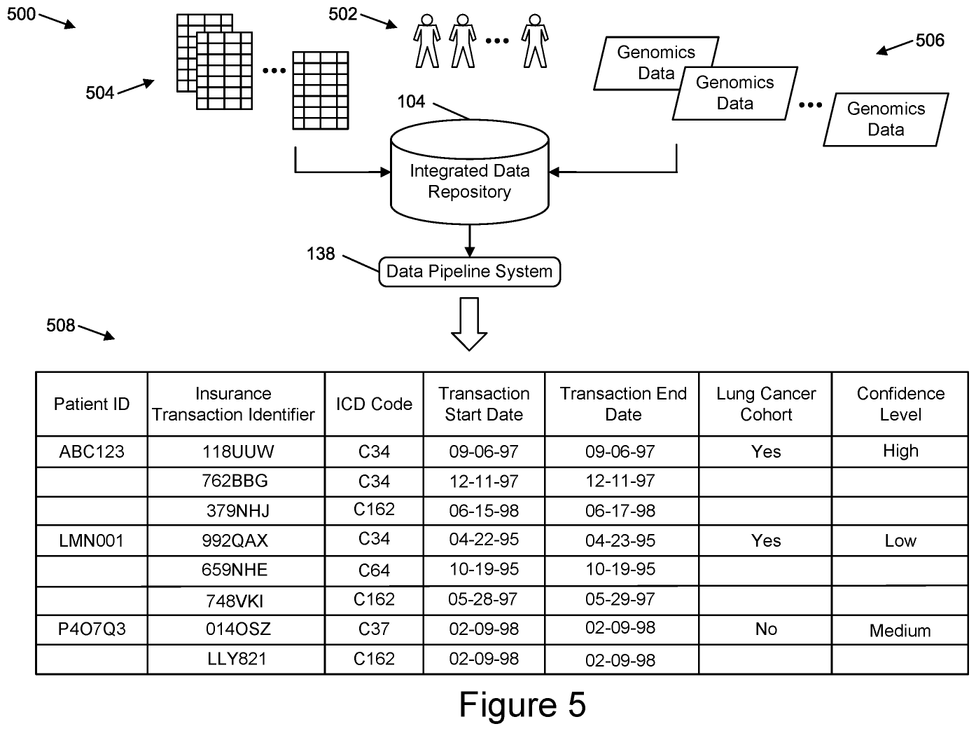

FIG. 5 illustrates a framework 500 to generate a dataset, by a data pipeline system 138, based on data stored by an integrated data repository 104, according to one or more implementations. The integrated data repository 104 may store health insurance claims data and genomics data for a group of individuals 502. For example, the integrated data repository 104 may store information obtained from health insurance claims records 504 of the group of individuals 502. For each individual included in the group of individuals 502, the integrated data repository 104 may store information obtained from multiple health insurance claim records 504. In various examples, the information stored by the integrated data repository 104 may include and/or be derived from thousands, tens of thousands, hundreds of thousands, up to millions of health insurance claims records 504. Additionally, each health insurance claim record may include multiple columns. As a result, the integrated data repository 104 may be generated through the analysis of millions of columns of health insurance claims data.

Further, although the health insurance claims data may be organized according to a structured data format, health insurance claims data is typically arranged to be viewed by health insurance providers, patients, and healthcare providers in order to show financial information and insurance code information related to services provided to individuals by healthcare providers. Thus, health insurance claims data is not easily analyzed to gain insights that may be available in relation to characteristics of individuals in which a biological condition is present and that may aid in the treatment of the individuals with respect to the biological condition. The integrated data repository 104 may be generated and organized by analyzing and modifying raw health insurance claims data in a manner that enables the data stored by the integrated data repository 104 to be further analyzed to determine trends, characteristics, features, and/or insights with respect to individuals in which one or more biological conditions may be present. Further, the integrated data repository 104 may be generated using genomics data records 506 of the group of individuals 502. In various examples, the large amounts of health insurance claims data may be matched with genomics data for the group of individuals 502 to generate the integrated data repository 104. In one or more examples, the processes and techniques implemented to integrate the health insurance claims records 504 and the genomics claims records 506 in order to generate the integrated data repository 104 may be complex and implement efficiency-enhancing techniques, systems, and processes in order to minimize the amount of computing resources used to generate the integrated data repository 104.

In one or more illustrative examples, the data pipeline system 138 may access information stored by the integrated data repository 104 to generate datasets that include a number of additional data records 508 that include information related to at least a portion of the group of individuals 502. In the illustrative example of FIG. 5, the additional data record 508 includes information indicating whether individuals are included in a cohort of individuals in which lung cancer is present. The data pipeline system 138 may execute a plurality of different sets of data processing instructions to determine a cohort of the group of individuals 502 in which lung cancer is present. In various examples, the additional data record 508 may indicate information used to determine a status of an individual 502 with respect to lung cancer, such as one or more transaction insurance identifier, one or more international classification of diseases (ICD) codes, and one or more health insurance transaction dates. In addition to including a column that indicates whether an individual 502 is included in the lung cancer cohort, the additional data record 508 may include a column indicating a confidence level of the status of the individual 502 with respect to the presence of lung cancer.

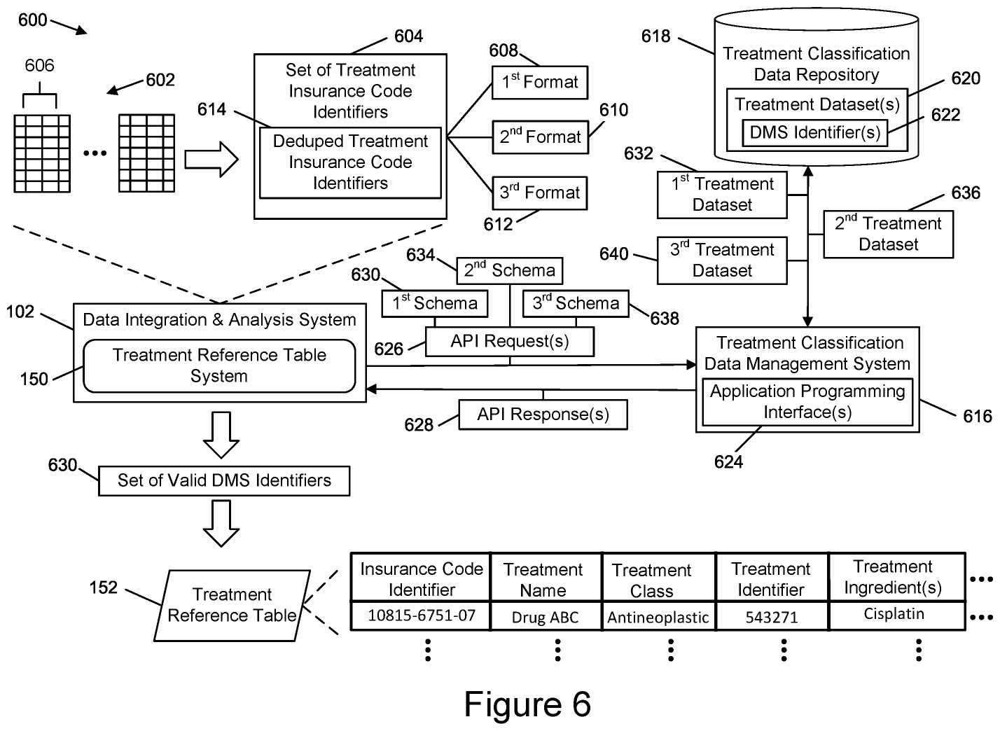

FIG. 6 illustrates an architecture 600 to generate a reference data table 152 indicating identifiers of treatments provided to patients in which one or more biological conditions may be present, according to one or more implementations. The architecture 600 can include the data integration and analysis system 102. The data integration and analysis system 102 can include the treatment reference table system 150 that can generate the treatment reference table 152. In one or more examples, the data integration and analysis system 102 can at least one of obtain or generate data tables 602 that include insurance code identifiers. The data tables 602 can include and/or be generated using information obtained from the health insurance claims data repository 106 of FIG. 1. In various examples, the data tables 602 can include one or more data tables stored by the integrated data repository 104 of FIG. 1. To illustrate, the data tables 602 can include a pharmacy records data table and/or a service lines data table stored by the integrated data repository 104.

The treatment reference system 150 can analyze the data tables 602 to generate a subset of insurance code identifiers included in the data tables 602. For example, the treatment reference system 150 can analyze the data tables 602 with respect to one or more criteria. In one or more examples, the treatment reference system 150 can analyze the information included in the data tables 602 to identify insurance code identifiers that correspond to treatments provided to individuals in which a biological condition is present. To illustrate, insurance code identifiers that are related to treatments can have one or more specified formats. In one or more illustrative examples, insurance code identifiers that correspond to treatments can have a specified number of alphanumeric characters or other symbols, such as 8 characters or symbols, 9 characters or symbols, 10 characters or symbols, 11 characters or symbols, 12 characters or symbols, and the like. Insurance code identifiers that correspond to treatments can also have one or more arrangements of alphanumeric symbols and/or characters.

In one or more additional illustrative examples, insurance code identifiers that correspond to treatments can have a number of segments with a number of alphanumeric characters and/or symbols included in individual segments. In various examples, an insurance code identifier that corresponds to treatments can have at least one segment, at least 2 segments, at least 3 segments, or at least 4 segments. Individual segments can include at least one alphanumeric symbol, at least 2 alphanumeric symbols, at least 3 alphanumeric symbols, or at least four alphanumeric symbols. In one or more examples, segments of insurance code identifiers corresponding to treatments can be separated by symbols. In various examples, segments of insurance code identifiers corresponding to treatments can be separated by at least one of dashes, commas, or periods.

In one or more implementations, the treatment reference table system 150 can analyze information included in the data tables 602 to determine values of columns and rows that correspond to the one or more criteria. The treatment reference table system 150 can produce a set of treatment insurance code identifiers 604 that include insurance code identifiers of the data tables 602 that satisfy the one or more criteria. For example, the treatment reference table system 150 can analyze the information stored by the data tables 602 to determine insurance code identifiers that correspond to one or more formats. In one or more illustrative examples, the treatment reference table system 150 can analyze information stored by the data tables 602 to determine whether insurance code identifiers correspond to formatting of at least one of NDC-9, NDC-10, or NDC-11. In various examples, at least tens of thousands of insurance code identifiers, hundreds of thousands of insurance code identifiers, up to millions of insurance code identifiers or more are analyzed by the treatment reference table system 150 to generate the set of treatment insurance code identifiers 604. In at least some examples, individual identifiers included in the set of treatment insurance code identifiers can uniquely identify a treatment provided to patients in which a biological condition is present.

In one or more additional examples, the treatment reference table system 150 can determine one or more columns of the data tables 602 that include insurance code identifiers that satisfy the one or more criteria. For example, the treatment reference table system 150 can determine that a number of columns 606 include insurance code identifiers that correspond to the formatting criteria of insurance code identifiers. In one or more examples, the number of columns 606 can be determined based on user input obtained by the data integration and analysis system 102. In one or more further examples, the treatment reference table system 150 can analyze information included in at least a portion of the columns of the data tables 602 to determine the number of columns 606 that include insurance code identifiers that satisfy one or more formatting criteria. In one or more illustrative examples, individual treatment insurance code identifiers included in the set of treatment code identifiers 604 can have a first format 608, a second format 610, and a third format 612. Although the illustrative example of FIG. 6 shows that the set of treatment insurance code identifiers 604 can have three formats, in additional implementations, the set of treatment insurance code identifiers 604 can have fewer formats or a greater number of formats.

The treatment reference table system 150 can also analyze the set of treatment insurance code identifiers 604 to determine a subset of treatment insurance code identifiers that includes one or more unique treatment insurance code identifiers. In one or more examples, the treatment reference table system 150 can perform one or more deduplication processes to produce deduped treatment insurance code identifiers 614 based on the set of treatment insurance code identifiers 604. In various examples, the deduped treatment insurance code identifiers 614 can include insurance code identifiers that correspond to treatments and that are different from each other insurance code identifier of the deduped treatment insurance code identifiers 614. To illustrate, individual unique treatment insurance code identifiers included in the deduped treatment insurance code identifiers 614 can include at least one alphanumeric character and/or other symbol located in at least one position that is not present in the corresponding position of other insurance code identifiers included in the deduped treatment insurance code identifiers 614.

The treatment reference table system 150 can analyze the deduped treatment insurance code identifiers 614 with respect to one or more additional format criteria. In various examples, the deduped treatment insurance code identifiers 614 can include unique treatment insurance code identifiers that are formatted according to one or more NDC formats and the treatment reference table system 150 can analyze the deduped treatment insurance code identifiers 614 to determine the NDC format of individual treatment insurance code identifiers included in the deduped treatment insurance code identifiers 614. In one or more examples, individual NDC formats can have one or more formatting characteristics that are different from one or more formatting characteristics of additional NDC formats. For example, insurance code identifiers formatted according to an NDC-9 format can have one or more first characteristics, insurance code identifiers formatting according to an NDC-10 format can have one or more second characteristics, and insurance code identifiers formatted according to an NDC-11 format can have one or more third characteristics. The one or more first characteristics can be different from the one or more second characteristics and the one or more third characteristics and the one or more second characteristics can be different from the one or more third characteristics. In one or more illustrative examples, the treatment reference table system 150 can determine a portion of the deduped treatment insurance code identifiers 614 that correspond to an NDC-9 format. Additionally, the treatment reference table system 150 can determine a portion of the deduped treatment insurance code identifiers 614 that correspond to an NDC-10 format. Further, the treatment reference table system 150 can determine a portion of the deduped treatment insurance code identifiers 614 that correspond to an NDC-11 format. In one or more additional illustrative examples, the treatment reference table system 150 can determine an NDC format of a treatment insurance code identifier based at least partly on determining a number of alphanumeric characters present in the treatment insurance code identifiers. In one or more further illustrative examples, the treatment reference table system 150 can determine an NDC format of a treatment insurance code identifier based at least partly on a number of segments included in the treatment insurance code identifier and/or a number of alphanumeric characters present in individual segments of the treatment insurance code identifier. In one or more scenarios, the first format 608 can correspond to an NDC-9 format, the second format 610 can correspond to an NDC-10 format, and the third format 612 can correspond to an NDC-11 format.

The data integration and analysis system 102 can obtain information from one or more reference information data repositories to obtain information that the treatment reference table system 150 uses to generate the treatment reference table 152. In various examples, individual treatment insurance code identifiers included in the deduped treatment insurance code identifiers 614 can be used to obtain information from one or more reference information data repositories. In one or more examples, the data integration and analysis system 102 can be in communication with a treatment classification data management system 616 via one or more communication networks. The treatment classification data management system 616 can be coupled to a treatment classification data repository 618. The treatment classification data management system 616 can manage the storage and retrieval of information stored by the treatment classification data repository 618. The treatment classification data repository 618 can store information related to treatment insurance code identifiers. In one or more illustrative examples, the treatment classification data repository 618 can store information related to treatments that correspond to treatment insurance code identifiers. To illustrate, the treatment classification data management system 616 can store one or more treatment datasets 620. In various examples, the treatment classification data management system 616 can be controlled, maintained, and implemented by an entity that is external to the entity that controls, maintains, and implements the data integration and analysis system 102. In one or more additional examples, the treatment classification data management system 616 can be internal with respect to the data integration and analysis system 102 and can be controlled, maintained, and implemented by a same entity as the data integration and analysis system 102. In these scenarios, the treatment classification data repository 618 can store copies of information obtained from an external data repository using one or more API requests.

An individual treatment dataset 620 can store information that corresponds to an individual treatment insurance code identifier. Individual treatment datasets 620 can correspond to an arrangement of data that can be accessed as a group in response to queries from the treatment classification data management system 616. In various examples, a treatment dataset 620 can include an additional identifier, such as a data management system (DMS) identifier 622, that is used by the treatment classification data management system 616 to store and retrieve information related to a treatment insurance code identifier. In one or more examples, an individual DMS identifier 622 used by the treatment classification data management system 616 can correspond to one or more treatment insurance code identifiers. In at least some scenarios, a DMS identifier 622 used by the treatment classification data management system 616 to store and retrieve information related to a treatment insurance code identifier can have a format that is different from a format of the treatment insurance code identifier. In one or more illustrative examples, the DMS identifiers 622 can include one or more RxNorm concept unique identifiers (RxCUIs).

In one or more implementations, a treatment dataset 620 can store information for a treatment insurance code identifier that corresponds to one or more names of one or more treatments that are related to the treatment insurance code identifier, one or more ingredients of one or more treatments related to the treatment insurance code identifier, one or more classes of one or more treatments related to the treatment insurance code identifier, one or more sources of the one or more classes, one or more term types of one or more treatments related to the treatment insurance code identifier, or one or more combinations thereof. In various examples, a treatment dataset 620 can store a status of a treatment that corresponds to a treatment insurance code identifier, at least one of a start date or an end date that a treatment insurance code identifier was active within the treatment classification data repository 618, a history of the treatment insurance code identifier in relation to the treatment classification data management system 616, or one or more combinations thereof. The status of a treatment insurance code identifier can indicate whether or not the treatment insurance code identifier is currently being used to identify information related to a respective treatment.

The treatment classification data management system 616 can implement one or more application programming interfaces (APIs) 624. The one or more APIs 624 can include calls that can be used to request information from the treatment classification data repository 618 by the treatment classification data management system 616. The calls of the one or more APIs 624 can include one or more fields and one or more formats to retrieve information stored by the treatment classification data repository 618. In one or more examples, the data integration and analysis system 102 can send one or more API requests 626 to the treatment classification data management system 616. The treatment classification data management system 616 can then generate queries to the treatment classification data repository 618 to retrieve data from the treatment classification data repository 618. In one or more illustrative examples, queries generated by the treatment classification data management system 616 can correspond to retrieving one or more treatment datasets 620 in response to an API request 626. The treatment classification data management system 616 can send one or more API responses 628 to the data integration and analysis system 102 based on the one or more API requests 626. The treatment reference table system 150 can generate at least a portion of the treatment reference table 152 using information included in the API responses 628.

In one or more examples, the API requests 626 can be generated according to one or more schema with individual schema being used to retrieve a specified set of data. In various examples, API requests 626 that correspond to a first schema 630 can be used to obtain a first treatment dataset 632 stored by the treatment classification data repository 618. In addition, API requests 626 that correspond to a second schema 634 can be used to obtain a second treatment dataset 636 stored by the treatment classification data repository 618. Further, API requests 626 that correspond to a third schema 638 can be used to obtain a third treatment dataset 640 stored by the treatment classification data repository 618.

In various examples, the treatment reference table system 150 can generate API requests 626 that correspond to the first schema 630 using a first set of information. Additionally, the treatment reference table system 150 can generate API requests 626 that correspond to the second schema 634 using a second set of information. Further, the treatment reference table system 150 can generate API requests 626 that correspond to the third schema 638 using a third set of information. For example, the treatment reference table system 150 can generate an API request 626 according to the first schema 630 using at least one of deduped treatment insurance code identifiers 614 having the first format 608 or deduped treatment insurance code identifiers 614 having the second format 610. In one or more examples, the treatment reference table system 150 can modify a deduped treatment insurance code identifier 614 having the first format 608 and/or a deduped treatment insurance code identifier 614 having the second format 610 to generate an API request 626 according to the first schema 630. To illustrate, the treatment reference table system 150 can at least one of add or remove one or more symbols and/or one or more alphanumeric characters from a deduped treatment insurance code identifier 614 having the first format 608 or from a deduped treatment insurance code identifier 614 having the second format 610 to generate an API request 626 according to the first schema 630. In one or more examples, the treatment reference table system 150 can add a hyphen to separate the last two alphanumeric characters of a deduped treatment insurance code identifier 614 having the first format 608 and/or the second format 610 to generate an API request 626 according to the first schema 630.

In one or more illustrative examples, the treatment reference table system 150 can generate an API request 626 according to the first schema 630 using a deduped treatment insurance code identifier 614 having an NDC-9 format or an NDC-10 format. For example, the treatment reference table system 150 can generate an API request 626 that includes the deduped treatment insurance code identifier 614 having the NDC-9 format or the NDC-10 format or a modified version of the deduped treatment insurance code identifier 614. The API request 626 can also include at least one of a command to retrieve information from the treatment classification data repository 618 or an identifier of a respective treatment dataset 620. Additional information can also be used to generate the API request 626 according to the first schema 630. The API request 626 can be formatted as a hypertext transfer protocol (HTTP) request.

The API request 626 generated by the treatment reference table system 150 can be sent to the treatment classification data management system 616. The treatment classification data management system 616 can then retrieve information from the treatment classification data repository 618 that corresponds to the first treatment dataset 632 and send the first treatment dataset 632 to the data integration and analysis system 102 via an API response 628. In various examples, the first treatment dataset 632 can include an additional treatment insurance code identifier having the NDC-11 format that corresponds to a deduped insurance treatment code identifier having the first format 608 or the second format 610. Thus, in these scenarios, an API request 626 generated according to the first schema 630 can be used to obtain a treatment insurance code identifier having an NDC-11 format that corresponds to a treatment insurance code identifier having an NDC-9 format or an NDC-10 format. In various examples, the treatment reference table system 150 can determine whether the additional treatment insurance code identifier included in the first treatment dataset 632 is included in the set of insurance code identifiers 604. In scenarios where the additional treatment insurance code identifier included in the first treatment dataset 632 is already included in the set of treatment insurance code identifiers 604, the additional treatment insurance code identifier can be ignored. In one or more additional examples, in situations where the additional treatment insurance code identifier included in the first treatment dataset 632 is not included in the set of treatment insurance code identifiers 604, the additional treatment insurance code identifier can be added to the deduped treatment insurance code identifiers 614.

The first treatment dataset 632 can include additional information. To illustrate, the first treatment dataset 632 can also include a DMS identifier 622 that corresponds to a deduped treatment insurance code identifier 614 used to generate the API request 626 according to the first schema 630. For example, the first treatment dataset 632 can include an RxCUI that corresponds to an NDC-9 identifier, an NDC-10 identifier, and/or an NDC-11 identifier. Additionally, the first treatment dataset 632 can include one or more properties of a treatment that corresponds to the deduped treatment insurance code identifier 614 used to generate an API request 626 according to the first schema 630. In various examples, the one or more properties can indicate packaging of the treatment, physical characteristics of the treatment (e.g., color, shape, etc.), dosing characteristics of the treatment, one or more additional characteristics of the treatment (e.g., generic, active status, inactive status, etc.), or one or more combinations thereof.

The treatment reference table system 150 can generate an API request 626 according to the second schema 634 using a deduped treatment insurance code identifier 614 having the third format 612. For example, the treatment reference table system 150 can generate an API request 626 according to the second schema 634 using a deduped treatment insurance code identifier 614 having an NDC-11 format. The API request 626 generated according to the second schema 634 can also include at least one of a command to retrieve information from the treatment classification data repository 618 or an identifier of a respective treatment dataset 620. Additional information can also be used to generate the API request 626 according to the second schema 634. The API request 626 can be formatted as a hypertext transfer protocol (HTTP) request.

In response to receiving an API request 626 generated according to the second schema 634 from the data integration and analysis system 102, the treatment classification data management system 616 can retrieve the second treatment dataset 636 from the treatment classification data repository 618 that corresponds to the deduped treatment insurance code identifier 614 used to generate the API request 626 having the third format 612. In various examples, the information included in the second treatment dataset 636 can indicate a status of the deduped treatment insurance code identifier 614 having the third format 612 within the treatment classification data management system 616. To illustrate, the second treatment dataset 636 can indicate whether or not a deduped treatment insurance code identifier 614 having an NDC-11 format can actively be used to retrieve information from the treatment classification data repository 618 using the deduped treatment insurance code identifier 614. In these scenarios, an API request 626 corresponding to the second schema 634 can be used to determine whether or not a deduped treatment insurance code identifier 614 having the third format 612 is valid.

In one or more examples, the treatment reference table system 150 can analyze the second treatment dataset 636 to determine a validity of a deduped treatment insurance code identifier 614 having the third format 612. For example, the treatment reference table system 150 can determine that the second treatment dataset 636 indicates that a deduped treatment insurance code identifier 614 having the third format 612 was not found in the treatment classification data repository 618. In these situations, the treatment reference table system 150 can determine that the deduped treatment insurance code identifier 614 having the third format 612 is not valid. Additionally, the treatment reference table system 150 can determine that the second treatment dataset 636 indicates that a similar, but not the same treatment insurance code identifier, is present in the treatment classification data repository 618. In these scenarios, the treatment reference table system 150 can also determine that the deduped treatment insurance code identifier 614 having the third format 612 is not valid. Further, the treatment reference table system 150 can determine that the second treatment dataset 636 indicates that information related to a deduped treatment insurance code identifier 614 having the third format 612 is proprietary. As a result, the treatment reference table system 150 can determine that the deduped treatment insurance code identifier 614 having the third format 612 is not valid. The treatment reference table system 150 can determine deduped treatment insurance code identifiers 614 that are valid according to one or more criteria and generate a set of valid deduped treatment insurance code identifiers. For individual valid deduped treatment insurance code identifiers, the treatment reference table system 150 can identify and extract a respective DMS identifier 622. The respective DMS identifiers 622 that correspond to valid deduped treatment insurance code identifiers 614 can be used to generate individual rows of the treatment reference table 152. In various examples, for deduped treatment insurance code identifiers 614 that are not valid, the treatment reference table system 150 can generate a row of the treatment reference table 152 and generate a comment indicating that the deduped treatment insurance code identifier 614 is not valid. In at least some scenarios, the treatment reference table system 150 can generate a comment indicating a reason that the deduped treatment insurance code identifier 614 is not valid, such as a similar treatment insurance code identifier having different packaging is present in the treatment classification data repository 618 or that the deduped treatment insurance code identifier has a classification of proprietary.

For each of the deduped treatment insurance code identifiers 614 that the treatment reference table system 150 determines to be valid, the treatment reference table system 150 can determine a DMS identifier 622 that corresponds to the valid deduped treatment insurance code identifier 614. The DMS identifier 622 can then be used to generate an API request 626 according to the third schema 638. In one or more illustrative examples, the API request 626 generated according to the third schema 638 can include an RxCUI extracted from at least one of the first treatment dataset 632 or the second treatment dataset 636. The API request 626 generated according to the third schema 638 can also include at least one of a command to retrieve information from the treatment classification data repository 618 or an identifier of a respective treatment dataset 620. Additional information can also be used to generate the API request 626 according to the third schema 638. The API request 626 can be formatted as a hypertext transfer protocol (HTTP) request.

In response to receiving an API request 626 generated according to the third schema 638 from the data integration and analysis system 102, the treatment classification data management system 616 can retrieve the third treatment dataset 640 from the treatment classification data repository 618. For example, the third treatment dataset 640 can be included in the API response 628 generated by the treatment classification data management system 616 based on an API request 626 corresponding to the third schema 638. In various examples, the information included in the third treatment dataset 640 can indicate one or more classes of treatments that correspond to one or more DMS identifiers 622 included in the API request 626 generated according to the third schema 638. For example, the information included in the third dataset 640 can include identifiers of classes, names of classes, types of classes, sources of classes, drug term types for treatments, names of treatments, or one or more combinations thereof In at least some examples, the treatment reference table system 150 can analyze the third treatment dataset 640 with respect to one or more criteria. To illustrate, the treatment reference table system 150 can analyze the third treatment dataset 640 to determine treatments associated with DMS identifiers 622 that are also associated with a term type related to treatments. Term types related to treatments can indicate at least one of ingredients of the treatments, classes of ingredients of treatments, dosages of treatments, forms of treatments (e.g., oral, drops), brand name of treatments, or synonyms of treatments.

The class information included in the third treatment dataset 640 can be used to determine one or more types of treatments with the one or more types of treatments having a respective source. The different sources of types of treatments can indicate various information related to treatments. In one or more examples, individual sources of treatment types can include different pieces of information about treatments. For example, a first source of treatment information can indicate pharmacological actions that correspond to treatments. Additionally, a second source of treatment information can include one or more mechanisms of action of treatments, a chemical structure and classification schema for chemicals or other ingredients included in treatments, and physiological effects of chemicals or ingredients included in treatments. The second source of treatment information can also include pharmacologic classes related to treatments, such as the United States Federal Drug Administration's established pharmacologic classes. A third source of treatment information can indicate groups of treatments that are determined based on the organ or organ system on which the treatments act. The third source of treatment information can also indicate chemical, pharmacological, and therapeutic properties of the treatments. Further, a fourth source of treatment information can indicate treatments according to the biological conditions being treated, the mechanism of action of the treatments, and the chemical structure of the treatments. The fourth source of treatment information can also indicate classifications of treatments and the effects of treatments on tissue, organs, and organ systems. Additionally, the fourth source of treatment information can indicate pharmacokinetics of treatments, such as the absorption, distribution, and elimination of active ingredients of treatments.

In one or more examples, the third treatment dataset 640 can be analyzed with respect to a prioritized list of sources of information about treatments. For example, the treatment reference table system 150 can analyze the third treatment dataset 640 according to a set of rules or protocols that analyze one or more fields of data included in the third treatment dataset 640. To illustrate, the rules implemented by the treatment reference table system 150 can cause the traversing of one or more specified fields of the third treatment dataset 640 to determine whether the one or more specified fields include a first identifier of a first source of treatment information having a first priority in the prioritized list of sources of treatment information. In situations where the treatment reference table system 150 determines that the first identifier is present in the one or more specified fields of the third treatment dataset 640, the treatment reference table system 150 can determine one or more first values of one or more columns of the treatment reference table 152 for the treatment. Additionally, in scenarios where the treatment reference table system 150 determines that the first identifier is not present in the one or more specified fields of the third treatment dataset 640, the treatment reference table system 150 can analyze the one or more specified fields with respect to a second source of treatment information included in the prioritized list of treatment information sources. The second source of treatment information can be associated with a second priority and a second identifier. In instances where the treatment reference table system 150 determines that the second identifier is present in the one or more specified fields of the third treatment dataset 640, the treatment reference table system 150 can determine one or more second values of one or more columns of the treatment reference table 152. In examples where the treatment reference table system 150 determines that the second identifier is not present in the one or more specified fields of the third treatment dataset 640, the treatment reference table system 150 can continue to analyze the one or more specified fields with respect to the prioritized list of sources of treatment information until a source of treatment information is identified that is included in the prioritized list of treatment information sources or until the treatment reference table system 150 determines that there are no treatment information sources included in the prioritized list that are present for a given treatment.

Based on the source of treatment information or the lack of the presence of a source of treatment information included in the prioritized list, the treatment reference table system 150 can determine respective values of one or more columns of the treatment reference table 152. The values determined by the treatment reference table system 150 for a given treatment can be different for different sources of treatment information. For example, the treatment reference table system 150 can determine one or more first values for one or more columns of the treatment reference table 152 in response to determining that a given treatment corresponds to a first source of the prioritized list of sources. In addition, the treatment reference table system 150 can determine one or more second values for one or more columns of the treatment reference table 152 in response to determining that a given treatment corresponds to a second source of the prioritized list of sources. In one or more illustrative examples, the treatment reference table system 150 can determine that the one or more first values that correspond to the first source of treatment information and/or the one or more second values that correspond to the second source of treatment information are related to at least one of a comments column of the treatment reference table 152, a treatment type column, a treatment class, a treatment category, or a treatment identifier.

The treatment reference table system 150 can generate the treatment reference table 150 based on information obtained from the data tables 602, the first treatment dataset 632, the second treatment dataset 636, and the third treatment dataset 640. For example, the treatment reference table system 150 can generate rows of the treatment reference table 152 that correspond to at least a portion of the individual treatments related to the deduped treatment insurance code identifiers 614. In one or more examples, the treatment reference table system 150 can analyze the data tables 602 to analyze the data tables 602 to determine insurance code identifiers of individual treatments and populate a column of the treatment reference table 152 with the insurance code identifiers of the treatments. Additionally, the treatment reference table system 150 can determine individual names of treatments, such as commercial names of treatments, by analyzing the second treatment dataset 636 and populate a column of the treatment reference table 152 using the names of the treatments. Further, the treatment reference table system 150 can determine individual classes and/or categories of treatments based on the third treatment dataset 640 and populate one or more columns of the treatment reference table 152 using the classes and/or categories. In various examples, the treatment reference table system 150 can determine ingredients of individual treatments by analyzing the second treatment dataset 636 and populate a column of the treatment reference table 152 using the ingredients of the treatments.

In one or more additional examples, the treatment reference table 152 can include at least one column that includes a comment or other miscellaneous information about individual treatments. The treatment reference table system 150 can determine values of a comments column by analyzing data included in at least one of the second treatment dataset 636 or the third treatment dataset 640. For example, the treatment reference table system 150 can determine a value of a comments column based on a source of treatment information for individual treatments included in the treatment reference table 152. To illustrate, the treatment reference table system 150 can determine a value of a comment column of the treatment reference table 152 by indicating a source of the treatment information or indicating a type of information associated with the source of treatment information. In one or more illustrative examples, the treatment reference table system 150 can determine that, for a given treatment, the source of information is a first source that includes a mechanism of action of the treatment. In these scenarios, the treatment reference table system 150 can determine the value of the comment column for the individual treatment to indicate that mechanism of action information is available for the treatment. In one or more additional examples, the treatment reference table system 150 can determine that, for a given treatment, the source of information is a second source that includes pharmacological class information. In these instances, the treatment reference table system 150 can determine the value of the comment column for the given treatment to indicate that pharmacological class information is available for the treatment.

In one or more further examples, the treatment reference table system 150 can determine that one or more values of columns of the treatment reference table 152 are to be set to null. To illustrate, the treatment reference table system 150 can determine that a source of information corresponding to a given treatment and determine that a value of a treatment source category and/or a value for a comment of a given treatment is to be set to null. In these situations, the treatment reference table system 150 can implement one or more rules or protocols indicating that one or more sources of treatment information correspond to a null value for a comment column. In addition, the treatment reference table system 150 can analyze at least one of the deduped treatment insurance code identifiers 614, the first treatment dataset 632, the second treatment dataset 636, or the third treatment dataset 640 and determine that at least one identifier corresponding to a treatment is not present. In these instances, the treatment reference table system 150 can determine that a value of a column for the treatment that corresponds to an identifier of the treatment, such as an insurance code identifier and/or a DMS identifier 622, is to be set to null.

In this way, the treatment reference table system 150 can analyze treatment data obtained from a number of disparate sources and generate the treatment reference table 152 using specified portions of the treatment data to generate values for the columns of the treatment reference table 152. As a result, the data stored by the treatment reference table 152 can be used by the data integration and analysis system 102 to determine information about treatments provided to one or more cohorts of individuals in which one or more specified biological conditions are present. For example, the data integration and analysis system 102 can analyze one or more values of one or more rows of the treatment reference table 152 to determine one or more insurance code identifiers that correspond to a name of the treatment or a class of the treatment. In one or more examples, the data integration and analysis system 102 can analyze values of a number of rows of the one or more data tables to determine one or more rows that include the one or more insurance code identifiers and determine one or more identifiers of individuals included in the one or more rows to produce a cohort of individuals that received the treatment in relation to the biological condition. In various examples, the data integration and analysis system 102 can determine genomics information of the cohort of individuals that received the treatment in relation to the biological condition and applying at least one of one or more statistical techniques or one or more machine learning techniques to determine one or more features of the cohort of individuals. In one or more illustrative examples, the one or more features can include at least one of a genetic mutation included in respective genomes of individuals included in the cohort of individuals, a genetic mutation of cell-free deoxyribonucleic acids (DNA) included in one or more samples obtained from individuals included in the cohort of individuals, an amount of cell-free DNA having the genetic mutation for the respective individuals included in the cohort of individuals, or a change in the amount of cell-free DNA having the genetic mutation for the respective individuals included in the cohort of individuals over a period of time.

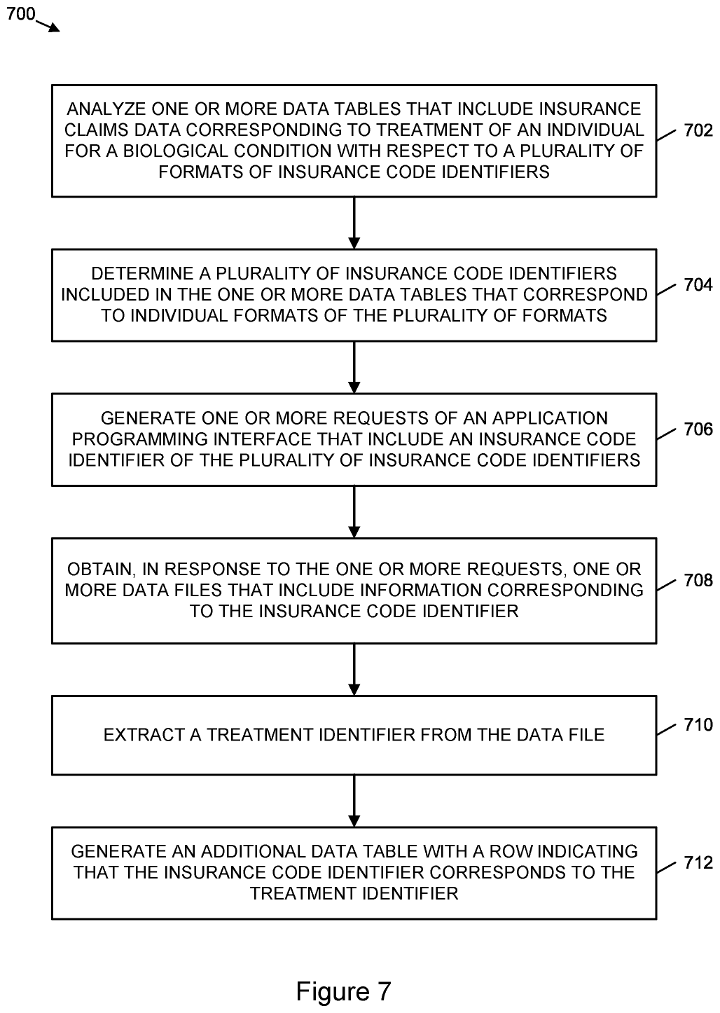

FIGS. 7, 8, and 9 illustrate example processes to generate an integrated data repository and generate datasets used in the analysis of information stored by the integrated data repository. The example processes are illustrated as collections of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

FIG. 7 is a flow diagram of an example process 700 to generate a treatment reference table that includes information about treatments provided to patients in which one or more biological conditions may be present, according to one or more implementations. At operation 702, the process 700 can include analyzing one or more data tables that include insurance claims data corresponding to treatment of an individual for a biological condition with respect to a plurality of formats of insurance code identifiers. The insurance claims data can include insurance code identifiers that correspond to a number of different interventions and/or services provided to individuals in relation to healthcare providers. In various examples, insurance code identifiers that correspond to different treatments can have different formats. For example, insurance code identifiers that correspond to pharmaceutical treatments can be formatted according to one or more NDC formats. Additionally, insurance code identifiers that correspond to medical procedures can be formatted according to one or more current procedural terminology (CPT) codes and/or one or more healthcare common procedure coding system (HCPCS) codes. Further, insurance code identifiers related to diagnosis of individuals with respect to biological conditions can correspond to one or more international classification of diseases (ICD) codes. In one or more examples, the insurance code identifiers can have at least a first format, a second format, and a third format. In one or more illustrative examples, the insurance code identifiers can have at least one of an NDC-9 format, an NDC-10 format, or an NDC-11 format.

At operation 704, the process 700 includes determining a plurality of insurance code identifiers included in the one or more data tables that correspond to individual formats of the plurality of formats. In one or more examples, the individual formats can correspond to an arrangement of at least one of alphanumeric characters or symbols. The arrangement of alphanumeric characters and/or symbols of an individual insurance code identifier can be analyzed with respect to the arrangements of alphanumeric characters and/or symbols of the individual formats. In response to determining at least a threshold similarity between an insurance code identifier and a respective insurance code identifier format, the insurance code identifier can be designated as having the respective format. In various examples, a first number of insurance code identifiers can be identified as having a first format, a second number of insurance code identifiers can be identified as having a second format, and a third number of insurance code identifiers can be identified as having a third format. In one or more illustrative examples, a first group of insurance code identifiers can have an NDC-9 format, a second group of insurance code identifiers can have an NDC-10 format, and a third group of insurance code identifiers can have an NDC-11 format.

Additionally, the process 700 can include, at operation 706, generating one or more requests of an application programming interface (API) that include an insurance code identifier included in the plurality of insurance code identifiers. The API request can include a string of at least one of alphanumeric characters or symbols that includes at least a portion of the insurance code identifier. In various examples, the insurance code identifier can be included in different API requests that can be used to retrieve different information from a data repository. For example, at least a portion of the insurance code identifier can be included in a first API request to obtain another version of the insurance code identifier having a different format. To illustrate, an API request can be generated using an insurance code identifier having an NDC-9 format or an NDC-10 format to retrieve a version of the insurance code identifier having an NDC-11 format. In one or more additional examples, the insurance code identifier can be used to generate a second API request that can be used to retrieve one or more identifiers of treatments associated with the insurance code identifier. In one or more implementations, the insurance code identifier can be used to generate an API request to retrieve a commercial name of a treatment, a standardized identifier used by the data repository to store information related to the treatment, such as an Rx Norm concept unique identifier (RxCUI), or both. In one or more further examples, the insurance code identifier can be used to generate an API request to retrieve a source of information related to a treatment that corresponds to the insurance code identifier and/or a category related to a treatment that corresponds to the insurance code identifier.

At operation 708, the process 700 can include obtaining, in response to the one or more API requests, one or more data files that include information corresponding to the insurance code identifier and, at operation 710, the process 700 can include extracting an identifier of a treatment from the data file. In one or more examples, the identifier of the treatment can include a name of a treatment, such as a commercial name of the treatment or a name of an ingredient of the treatment. In one or more additional examples, the identifier of the treatment can include an RxCUI of the treatment.

Further, the process 700 can include, at operation 712, generating an additional data table with a row indicating that the insurance code identifier corresponds to the identifier of the treatment. In one or more examples, the additional data table can have a number of rows with each row corresponding to a single treatment. In various examples, the treatments included in the additional data table can be used to treat a biological condition. The additional data table can also include a number of columns that have values corresponding to information related to the respective treatments. In one or more examples, the additional data table can include a column that includes the insurance code identifier of the treatment and another column that includes the treatment identifier obtained from the data repository. In one or more illustrative examples, a row corresponding to the treatment can have a value of a first column that corresponds to an identifier having an NDC-9 format, an NDC-10 format, or an NDC-11 format and a second column that corresponds to an RxCUI of the treatment. The row corresponding to the treatment can also include a third column that includes a name of the treatment. Additional columns of the additional data table can include values that correspond to a source of information about the treatment, a category of the treatment, a status of the treatment, dates when the treatment was actively used, or one or more combinations thereof.

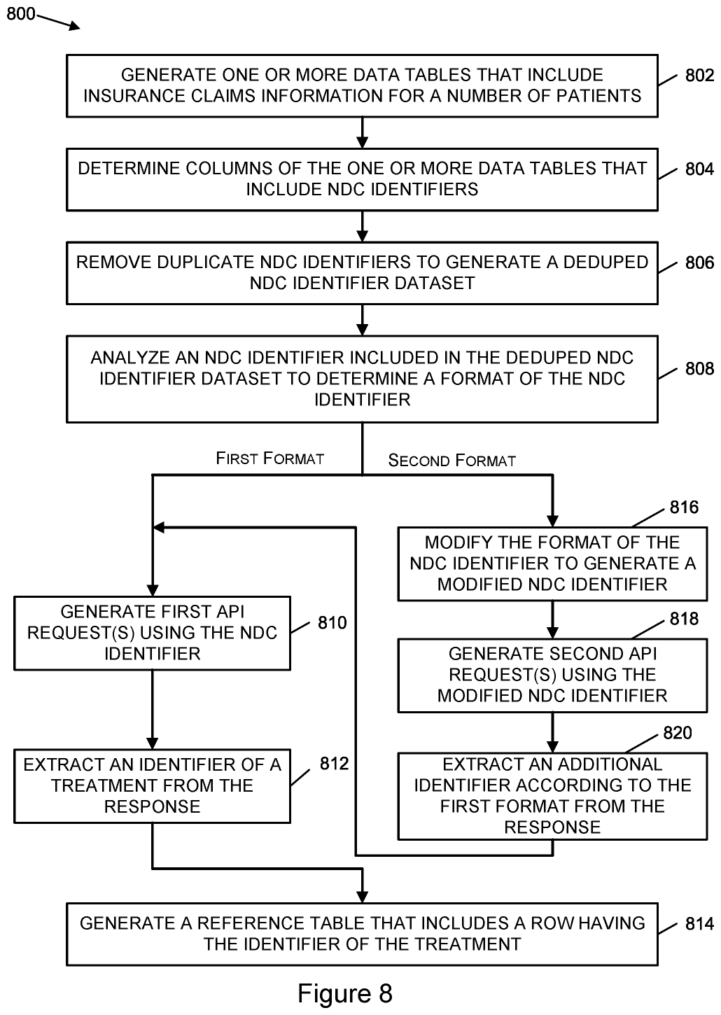

FIG. 8 is a flow diagram of an example process 800 to determine an identifier of a drug that corresponds to an insurance code identifier using one or more application programming interface (API) requests, according to one or more implementations. The process 800 can include, at operation 802, generating one or more data tables that include insurance claims information for a number of patients. The one or more data tables can include information obtained from a data repository storing health insurance claims data, such as the health insurance claims data repository 106 of FIG. 1. At operation 804, the process 800 can include determining one or more columns of the one or more data tables that include identifiers having an NDC format. In various examples, the one or more data tables can be arranged such that NDC identifiers are present in one or more specified columns of the one or more data tables. In one or more additional examples, values of columns of the one or more data tables can be analyzed to identify values of columns having one or more formats that correspond to NDC identifiers. The one or more formats can include at least one of an NDC-9 format, an NDC-10 format, or an NDC-11 format. The identifiers having an NDC format can each correspond to a treatment for at least one biological condition. In one or more illustrative examples, the treatment can include a pharmaceutical that can treat one or more biological conditions.

In addition, the process 800 can include, at operation 806, removing duplicate identifiers having an NDC format to generate a dataset including deduped identifiers having an NDC format. Further, at operation 808, the process 800 can include analyzing an identifier having an NDC format included in the dataset to determine a respective format of the identifier. In various examples, the NDC identifiers included in the deduped NDC identifier dataset can be grouped according to the NDC format of the identifiers. For example, a first set of identifiers having a first NDC format can be included in a first group of identifiers, a second set of identifiers having a second NDC format can be included in a second group of identifiers, and a third set of identifiers having a third NDC format can be included in a third group of identifiers.

In situations where the NDC identifier has a first format, the process 800 can move to 810 where one or more first API requests are generated using the NDC identifier. In at least some examples, the first format can include an NDC-11 format. The one or more API requests can be used to retrieve information from one or more datasets. The information obtained using the one or more API requests can include at least one of a source of the NDC identifier, a status of the treatment corresponding to the NDC identifier, a start date when the NDC identifier was activated, an end date when the NDC identifier was no longer active, one or more names of the treatment corresponding to the NDC identifier, or additional information about the NDC identifier. In one or more examples, the information obtained using the one or more API requests can include an additional identifier of the treatment that corresponds to the initial NDC identifier. At operation 812, the process 800 can include extracting the additional identifier from a response to the one or more first API requests. The additional identifier can be assigned to the treatment and/or the NDC identifier by a third-party. In one or more examples, the additional identifier can be unique with respect to identifiers of other treatments assigned by the third party. In one or more illustrative examples, the additional identifier can include an RxCUI of the treatment.

The process 800 can include, at operation 814, generating a reference table that includes a row having the identifier of the treatment. The reference table can include a plurality of rows with individual rows of the plurality of rows corresponding to an individual treatment. The reference table can also include a number of columns with values having information about the individual treatments. For example, the reference table can include columns with values indicating one or more categories of treatments, one or more additional identifiers of treatments, a status of treatments, ingredients of treatments, one or more combinations thereof, and the like.

In scenarios where the outcome of operation 808 indicates that the format of the NDC identifier corresponds to a second format, the process 800 can proceed to operation 816 from operation 808. Operation 816 can include modifying the format of the NDC identifiers to generate a modified NDC identifier. In one or more examples, the second format can correspond to an NDC-9 format or an NDC-10 format. In various examples, modifying the NDC identifier can include removing one or more alphanumeric characters or symbols from the NDC identifier. Additionally, modifying the NDC identifier can include adding one or more alphanumeric characters or symbols to the NDC identifier. In one or more illustrative examples, the NDC identifier can be modified by adding a dash symbol prior to the last two digits of the NDC identifier. In one or more additional illustrative examples, the NDC identifier can be modified by adding one or more zeros to one or more segments of the NDC identifier. In one or more further examples, the NDC identifier can be modified by removing one or more alphanumeric characters and/or symbols from the NDC identifier.

At operation 818, the process 800 can include generating one or more second API requests using the modified NDC identifier. The one or more second API requests can be used to obtain information from an additional dataset. The additional dataset can include a number of different identifiers related to the initial NDC identifier having the second format. For example, the additional dataset can include an additional identifier having the first format. To illustrate, the NDC identifier can have an NDC-9 format or an NDC-10 format and the additional identifier can have an NDC-11 format. At operation 824, the process 800 can include extracting the additional NDC identifier having the first format from the response to the one or more second API requests. Based on the additional NDC identifier having the first format, the process can move to 810 where the one or more first API requests can be generated using the additional NDC identifier having the first format and proceed to operation 812 and operation 814 where the treatment reference table is generated using information obtained using one or more first API requests that are generated based on the additional identifier having the first format.

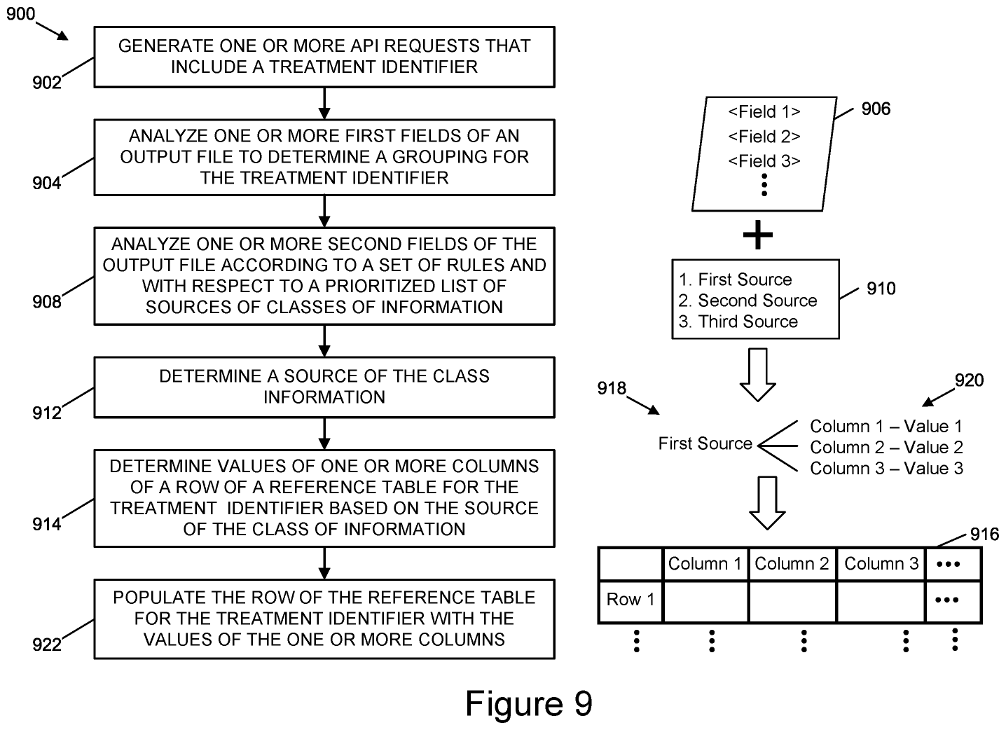

FIG. 9 is a flow diagram of an example process 900 to determine a class corresponding to an identifier of a treatment and to include information related to the class in a reference data table that includes the identifier of the treatment, according to one or more implementations. At operation 902, the process 900 can include generating one or more API requests that include a treatment identifier. In one or more examples, the treatment identifier can correspond to an identifier obtained from a data repository that stores information about a number of treatments. In one or more illustrative examples, the treatment identifier can include an RxCUI of a treatment. The one or more API requests can be used to obtain a specific set of information from the data repository. The one or more API requests can have a format and/or structure that is different from the format and/or structure of other API requests. The one or more API requests can be used to retrieve information stored at a respective storage location.

At 904, the process 900 can include analyzing one or more first fields of an output file 906 to determine a grouping for the treatment identifier. In one or more examples, in response to the one or more API requests an output file can be received that includes a number of fields and respective values for the one or more fields. In various examples, the output file 906 can include a field that indicates at least one of an identifier of a class that corresponds to the treatment identifier, an identifier of a type of the class that corresponds to the treatment identifier, or a source of a class relations or other information that corresponds to the treatment identifier. In one or more additional examples, the output file 906 can include fields having values that correspond to a name of a treatment related to the treatment identifier and/or a term type of the treatment that corresponds to the treatment identifier.

The process 900 can also include, at operation 908, analyzing one or more second fields of the output file 906 according to a set of rules and with respect to a prioritized list 910 of sources of class of information. The prioritized list 910 can indicate an ordered number of sources of classes of information for a treatment identifier. In scenarios where the RxNORM database is being queried, the prioritized list can include sources of treatment-class relations, such as Anatomical Therapeutic Chemical (ATC), Food and Drug Administration Structured Product Labeling (FDASPL), Federal Medication Terminologies Subject Matter Expert (FMTSME), Medication Reference Terminology (MEDRT), Medical Subject Headings (MeSH), RxNorm (by the National Library of Medicine), or SNOMEDCT (by the International Health Terminology Standards Development Organization). The values for a field of the output file 906 that correspond to a class of information can be analyzed with respect to the prioritized list 910 to, at operation 912, determine a source of the class of information, such as treatment class-relation information. The values for a field of the output file 906 that corresponds to the class of information of the treatment identifier can also be used to, at operation 914, determine values of one or more columns of a row of a reference table 916 for the treatment identifier based on the source of the class of information. For example, the values of a field of the output file 906 that corresponds to the source of class information for the treatment identifier can be used to determine the source of the class information for the treatment identifier. In one or more illustrative examples, the class name for the treatment identifier can be “pain” and the class type can be “disease”. The source of the class information can indicate a mechanism of action by which the treatment functions or a pharmacological class related to the treatment. The source of the class information can also indicate a chemical structure of one or more ingredients of the treatment and/or pharmacokinetics information related to the treatment.

In the illustrative example of FIG. 9, the source of the class information for the treatment can be a first source 918 that corresponds to values 920. At operation 922, the values 920 can be used to populate the row of the reference table 916. That is, for individual sources of class information for a treatment, respective values for columns of the reference table 916 that correspond to the treatment can be determined. For example, a first column can be populated with a name of the first source 918 and a second column can be populated with a type related to the first source 918. Additionally, values related to a third column that corresponds to a comment related to the treatment can be populated according to the values 920. In various examples, a comment can indicate whether the first source 918 includes mechanism of action information or pharmacological class information for a treatment.

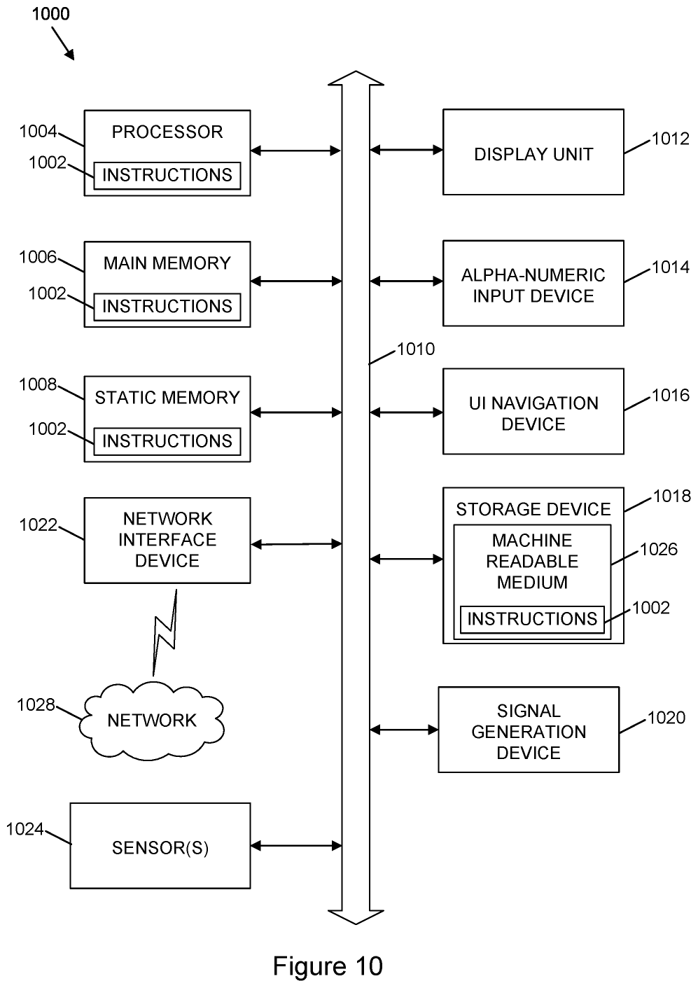

FIG. 10 illustrates a diagrammatic representation of a machine 1000 in the form of a computer system within which a set of instructions may be executed for causing the machine 1000 to perform any one or more of the methodologies discussed herein, according to an example, according to an example implementation. Specifically, FIG. 10 shows a diagrammatic representation of the machine 1000 in the example form of a computer system, within which instructions 1002 (e.g., software, a program, an application, an applet, an app, or other executable code) for causing the machine 1000 to perform any one or more of the methodologies discussed herein may be executed. For example, the instructions 1002 may cause the machine 1000 to implement the architectures and frameworks 100, 200, 300, 400, 500, 600 described with respect to FIGS. 1, 2, 3, 4, 5, and 6, respectively, and to execute the methods 700, 800, 900 described with respect to FIGS. 7, 8, and 9, respectively.

The instructions 1002 transform the general, non-programmed machine 1000 into a particular machine 1000 programmed to carry out the described and illustrated functions in the manner described. In alternative implementations, the machine 1000 operates as a standalone device or may be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1002, sequentially or otherwise, that specify actions to be taken by the machine 1000. Further, while only a single machine 1000 is illustrated, the term “machine” shall also be taken to include a collection of machines 1000 that individually or jointly execute the instructions 1002 to perform any one or more of the methodologies discussed herein.

Examples of machine 1000 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In implementations in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service”

(SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example implementations (e.g., apparatus, systems, or methods) can be implemented in digital electronic circuitry, in computer hardware, in firmware, in software, or in any combination thereof. Example implementations can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., computing device 800) and software architectures that can be deployed in example implementations.

In an example, the machine 1000 can operate as a standalone device or the machine 1000can be connected (e.g., networked) to other machines.

In a networked deployment, the machine 1000 can operate in the capacity of either a server or a client machine in server-client network environments. In an example, machine 1000can act as a peer machine in peer-to-peer (or other distributed) network environments. The machine 1000 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the computing device 800. Further, while only a single machine 1000 is illustrated, the term “computing device” shall also be taken to include any collection of machines that individually or jointly execute a set (or multiple sets) of instructions to perform any one or more of the methodologies discussed herein.

Example machine 1000 can include a processor 1004 (e.g., a central processing unit CPU), a graphics processing unit (GPU) or both), a main memory 1006 and a static memory 1008, some or all of which can communicate with each other via a bus 1010. The machine 1000 can further include a display unit 1012, an alphanumeric input device 1014 (e.g., a keyboard), and a user interface (UI) navigation device 1016 (e.g., a mouse). In an example, the display unit 1012, input device 1014 and UI navigation device 1016 can be a touch screen display. The machine 1000 can additionally include a storage device (e.g., drive unit) 1018, a signal generation device 1020 (e.g., a speaker), a network interface device 1022, and one or more sensors 1024, such as a global positioning system (GPS) sensor, compass, accelerometer, or another sensor.

The storage device 1018 can include a machine readable medium 1026 on which is stored one or more sets of data structures or instructions 1002 (e.g., software) embodying or utilized by any one or more of the methodologies or functions described herein. The instructions 1002 can also reside, completely or at least partially, within the main memory 806, within static memory 1008, or within the processor 1004 during execution thereof by the computing device 800. In an example, one or any combination of the processor 1004, the main memory 1006, the static memory 1008, or the storage device 1018 can constitute machine readable media.

While the machine readable medium 1026 is illustrated as a single medium, the term “machine readable medium” can include a single medium or multiple media (e.g., a centralized or distributed database, and/or associated caches and servers) that configured to store the one or more instructions 1002. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory

(EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1002 can further be transmitted or received over a communications network 1028 using a transmission medium via the network interface device 1022 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Some implementations are described as numbered examples (Example 1, 2, 3, etc.). These are provided as examples only and do not limit the technology disclosed herein.

Aspect 1. A method comprising: analyzing, by a computing system including processing circuitry and memory, one or more data tables stored by a data repository with respect to one or more criteria, the one or more data tables including insurance claims data corresponding to one or more treatments provided to an individual for a biological condition and the one or more criteria indicating one or more formats of insurance code identifiers; determining, by the computing system, a plurality of insurance code identifiers included in the one or more data tables that corresponds to the one or more criteria, analyzing, by the computing system, a subset of the plurality of insurance code identifiers to determine one or more formats of the subset of the plurality of insurance code identifiers; generating, by the computing system, one or more requests of an application programming interface (API) calls that include an insurance code identifier of the subset of the plurality of insurance code identifiers; obtaining, by the computing system and in response to the one or more requests of the API, a data file that includes information corresponding to the insurance code identifier; extracting, by the computing system, a treatment identifier from the data file; and generating, by the computing system, an additional data table with a row indicating that the insurance code identifier corresponds to the treatment identifier.

Aspect 2. The method of aspect 1, comprising determining, by the computing system, one or more columns of the one or more data tables that include insurance code identifiers corresponding to treatment of individuals.

Aspect 3. The method of aspect 1 or 2, comprising: determining, by the computing system, that a first insurance code identifier of the plurality of insurance code identifiers is a duplicate of a second insurance code identifier of the plurality of insurance code identifiers; and removing, by the computing system, the second insurance code identifier from the plurality of insurance code identifiers to produce the plurality of insurance code identifiers.

Aspect 4. The method of any one of aspects 1-3, wherein the plurality of insurance code identifiers correspond to a plurality of National Drug Code (NDC) identifiers.

Aspect 5. The method of any one of aspects 1-4, comprising: identifying, by the computing system, a first insurance code identifier of the subset of the plurality of insurance code identifiers; determining, by the computing system, that the first insurance code identifier corresponds to a first format of insurance code identifiers; and modifying, by the computing system, the first insurance code identifier to produce a second insurance code identifier that corresponds to a second format of insurance code identifiers.

Aspect 6. The method of aspect 5, comprising: generating, by the computing system, one or more additional requests of an additional API that include the second insurance code identifier; obtaining, by the computing system and in response to the one or more additional calls of the additional API, an additional data file that includes information corresponding to the third insurance code identifier; and extracting, by the computing system, the insurance code identifier from the additional data file.

Aspect 7. The method of any one of aspects 1-4, comprising: generating, by the computing system, one or more additional calls of the API that include an additional insurance code identifier; obtaining, by the computing system and in response to the one or more additional calls of the API, an additional data file that includes additional information corresponding to the additional insurance code identifier; and determining, by the computing system, that at least one valid insurance code identifier is not included in the additional information.

Aspect 8. The method of any one of aspects 1-7, comprising: generating, by the computing system, one or more additional calls of the API that include an additional insurance code identifier of the subset of the plurality of insurance code identifiers; obtaining, by the computing system and in response to the one or more additional calls of the API, an additional data file that includes additional information corresponding to the additional insurance code identifier; analyzing, by the computing system, the additional information with respect to one or more additional criteria; and determining, by the computing system, that the additional information does not include at least one drug identifier.

Aspect 9. The method of any one of aspects 1-8, comprising: generating, by the computing system, one or more additional calls of an additional API that includes the treatment identifier; obtaining, by the computing system and in response to the one or more additional calls, an additional data file that includes additional information corresponding to the drug identifier; and determining, by the computing system and based on the additional information, a class of drugs that corresponds to the drug identifier.

Aspect 10. The method of any one of aspects 1-9, comprising: determining, by the computing system and based on the additional information, a source of the class of drugs analyzing, by the computing system, the source of the class of drugs with respect to a prioritized number of drug sources; extracting, by the computing system and from one or more fields of the data file, at least a portion of the additional information; and adding, by the computing system, the at least a portion of the additional information to the row of the database table.

Aspect 11. The method of aspect 10, wherein the prioritized number of drug sources includes a first source of the class of drugs having a first priority and a second source of the class of drugs having a second priority that is lower than the first priority.

Aspect 12. The method of aspect 10 or 11, comprising: analyzing, by the computing system, the additional information with respect to the first source of the class of drugs; determining, by the computing system, that the additional information does not include a source of the class of drugs that corresponds to the first class; analyzing, by the computing system, the additional information with respect to the second sources of the class of drugs; and extracting, by the computing system, at least a portion of the additional information from the additional data file in relation to the second source of the class of drugs.

Aspect 13. The method of any one of aspects 1-12, comprising: receiving, by the computing system, a request to identify a group of individuals that received a treatment in response to a biological condition being present with respect to the group of individuals, wherein the request includes a name of the treatment or a class of the treatment; analyzing, by the computing system, one or more values of one or more rows of the additional data table to determine one or more insurance code identifiers that correspond to the name of the treatment or the class of the treatment; analyzing, by the computing system, values of a number of rows of the one or more data tables to determine one or more rows that include the one or more insurance code identifiers; and determining, by the computing system, one or more identifiers of individuals included in the one or more rows to produce a cohort of individuals that received the treatment in relation to the biological condition.

Aspect 14. The method of aspect 13, comprising: determining, by the computing system, genomics information of the cohort of individuals that received the treatment in relation to the biological condition; and applying, by the computing system, at least one of one or more statistical techniques or one or more machine learning techniques to determine one or more features of the cohort of individuals.

Aspect 15. The method of aspect 14, wherein the one or more features include at least one of a genetic mutation included in respective genomes of individuals included in the cohort of individuals, a genetic mutation of cell-free deoxyribonucleic acids (DNA) included in one or more samples obtained from individuals included in the cohort of individuals, an amount of cell-free DNA having the genetic mutation for the respective individuals included in the cohort of individuals, or a change in the amount of cell-free DNA having the genetic mutation for the respective individuals included in the cohort of individuals over a period of time.

Aspect 16. A system comprising: one or more hardware processing units; one or more computer-readable storage media storing computer-executable instructions that, when executed by the one or more hardware processing units, cause the system to perform operations comprising: analyzing one or more data tables stored by a data repository with respect to one or more criteria, the one or more data tables including insurance claims data corresponding to one or more treatments provided to an individual for a biological condition and the one or more criteria indicating one or more formats of insurance code identifiers; determining a plurality of insurance code identifiers included in the one or more data tables that corresponds to the one or more criteria, analyzing a subset of the plurality of insurance code identifiers to determine one or more formats of the subset of the plurality of insurance code identifiers; generating one or more requests of an application programming interface (API) calls that include an insurance code identifier of the subset of the plurality of insurance code identifiers; obtaining, in response to the one or more requests of the API, a data file that includes information corresponding to the insurance code identifier; extracting a treatment identifier from the data file; and generating an additional data table with a row indicating that the insurance code identifier corresponds to the treatment identifier.

Aspect 17. The system of aspect 16, wherein the one or more computer-readable storage media store additional computer-executable instructions that, when executed by the one or more hardware processing units, cause the system to perform additional operations comprising determining one or more columns of the one or more data tables that include insurance code identifiers corresponding to treatment of individuals.

Aspect 18. The system of aspect 16 or 17, wherein the one or more computer-readable storage media store additional computer-executable instructions that, when executed by the one or more hardware processing units, cause the system to perform additional operations comprising: determining that a first insurance code identifier of the plurality of insurance code identifiers is a duplicate of a second insurance code identifier of the plurality of insurance code identifiers; and removing the second insurance code identifier from the plurality of insurance code identifiers to produce the plurality of insurance code identifiers.

Aspect 19. The system of any one of aspects 16-18, wherein the plurality of insurance code identifiers correspond to a plurality of National Drug Code (NDC) identifiers.

Aspect 20. The system of any one of aspects 16-19, wherein the one or more computer-readable storage media store additional computer-executable instructions that, when executed by the one or more hardware processing units, cause the system to perform additional operations comprising: identifying a first insurance code identifier of the subset of the plurality of insurance code identifiers; determining that the first insurance code identifier corresponds to a first format of insurance code identifiers; and modifying the first insurance code identifier to produce a second insurance code identifier that corresponds to a second format of insurance code identifiers.

Aspect 21. The system of aspect 20, wherein the one or more computer-readable storage media store additional computer-executable instructions that, when executed by the one or more hardware processing units, cause the system to perform additional operations comprising: generating one or more additional requests of an additional API that include the second insurance code identifier; and obtaining, in response to the one or more additional calls of the additional API, an additional data file that includes information corresponding to the third insurance code identifier; and extracting the insurance code identifier from the additional data file.

Aspect 22. The system of any one of aspects 16-19, wherein the one or more computer-readable storage media store additional computer-executable instructions that, when executed by the one or more hardware processing units, cause the system to perform additional operations comprising: generating one or more additional calls of the API that include an additional insurance code identifier; obtaining, in response to the one or more additional calls of the API, an additional data file that includes additional information corresponding to the additional insurance code identifier; and determining that at least one valid insurance code identifier is not included in the additional information.

Aspect 23. The system of any one of aspects 16-22, wherein the one or more computer-readable storage media store additional computer-executable instructions that, when executed by the one or more hardware processing units, cause the system to perform additional operations comprising: generating, by the computing system, one or more additional calls of the API that include an additional insurance code identifier of the subset of the plurality of insurance code identifiers; obtaining, in response to the one or more additional calls of the API, an additional data file that includes additional information corresponding to the additional insurance code identifier; analyzing the additional information with respect to one or more additional criteria; and determining that the additional information does not include at least one drug identifier.

Aspect 24. The system of any one of aspects 16-23, wherein the one or more computer-readable storage media store additional computer-executable instructions that, when executed by the one or more hardware processing units, cause the system to perform additional operations comprising: generating one or more additional calls of an additional API that includes the treatment identifier; obtaining, in response to the one or more additional calls, an additional data file that includes additional information corresponding to the drug identifier; and determining, based on the additional information, a class of drugs that corresponds to the drug identifier.

Aspect 25. The system of any one of aspects 16-24, wherein the one or more computer-readable storage media store additional computer-executable instructions that, when executed by the one or more hardware processing units, cause the system to perform additional operations comprising: determining, based on the additional information, a source of the class of drugs analyzing the source of the class of drugs with respect to a prioritized number of drug sources; extracting, from one or more fields of the data file, at least a portion of the additional information; and adding the at least a portion of the additional information to the row of the database table.

Aspect 26. The system of aspect 25, wherein the prioritized number of drug sources includes a first source of the class of drugs having a first priority and a second source of the class of drugs having a second priority that is lower than the first priority.

Aspect 27. The system of aspect 25 or 26, wherein the one or more computer-readable storage media store additional computer-executable instructions that, when executed by the one or more hardware processing units, cause the system to perform additional operations comprising: analyzing the additional information with respect to the first source of the class of drugs; determining that the additional information does not include a source of the class of drugs that corresponds to the first class; analyzing the additional information with respect to the second sources of the class of drugs; and extracting at least a portion of the additional information from the additional data file in relation to the second source of the class of drugs.

Aspect 28. The system of any one of aspects 16-27, wherein the one or more computer-readable storage media store additional computer-executable instructions that, when executed by the one or more hardware processing units, cause the system to perform additional operations comprising: receiving a request to identify a group of individuals that received a treatment in response to a biological condition being present with respect to the group of individuals, wherein the request includes a name of the treatment or a class of the treatment; analyzing one or more values of one or more rows of the additional data table to determine one or more insurance code identifiers that correspond to the name of the treatment or the class of the treatment; analyzing values of a number of rows of the one or more data tables to determine one or more rows that include the one or more insurance code identifiers; and determining one or more identifiers of individuals included in the one or more rows to produce a cohort of individuals that received the treatment in relation to the biological condition.

Aspect 29. The system of aspect 28, wherein the one or more computer-readable storage media store additional computer-executable instructions that, when executed by the one or more hardware processing units, cause the system to perform additional operations comprising: determining genomics information of the cohort of individuals that received the treatment in relation to the biological condition; and applying at least one of one or more statistical techniques or one or more machine learning techniques to determine one or more features of the cohort of individuals.

Aspect 30. The system of aspect 29, wherein the one or more features include at least one of a genetic mutation included in respective genomes of individuals included in the cohort of individuals, a genetic mutation of cell-free deoxyribonucleic acids (DNA) included in one or more samples obtained from individuals included in the cohort of individuals, an amount of cell-free DNA having the genetic mutation for the respective individuals included in the cohort of individuals, or a change in the amount of cell-free DNA having the genetic mutation for the respective individuals included in the cohort of individuals over a period of time.

Aspect 31. One or more non-transitory computer-readable storage media storing computer-executable instructions that, when executed by one or more hardware processing units, cause a system to perform operations comprising: analyzing one or more data tables stored by a data repository with respect to one or more criteria, the one or more data tables including insurance claims data corresponding to one or more treatments provided to an individual for a biological condition and the one or more criteria indicating one or more formats of insurance code identifiers; determining a plurality of insurance code identifiers included in the one or more data tables that corresponds to the one or more criteria, analyzing a subset of the plurality of insurance code identifiers to determine one or more formats of the subset of the plurality of insurance code identifiers; generating one or more requests of an application programming interface (API) calls that include an insurance code identifier of the subset of the plurality of insurance code identifiers; obtaining, in response to the one or more requests of the API, a data file that includes information corresponding to the insurance code identifier; extracting a treatment identifier from the data file; and generating an additional data table with a row indicating that the insurance code identifier corresponds to the treatment identifier.

Aspect 32. The one or more non-transitory computer-readable media of aspect 31, comprising additional computer-executable instructions that, when executed by the one or more hardware processing units, cause the system to perform additional operations comprising determining one or more columns of the one or more data tables that include insurance code identifiers corresponding to treatment of individuals.

Aspect 33. The one or more non-transitory computer-readable media of aspect 31 or 32, comprising additional computer-executable instructions that, when executed by the one or more hardware processing units, cause the system to perform additional operations comprising: determining that a first insurance code identifier of the plurality of insurance code identifiers is a duplicate of a second insurance code identifier of the plurality of insurance code identifiers; and removing the second insurance code identifier from the plurality of insurance code identifiers to produce the plurality of insurance code identifiers.

Aspect 34. The one or more non-transitory computer-readable media of any one of aspects 31-33, wherein the plurality of insurance code identifiers correspond to a plurality of National Drug Code (NDC) identifiers.

Aspect 35. The one or more non-transitory computer-readable media of any one of aspects 31-34, comprising additional computer-executable instructions that, when executed by the one or more hardware processing units, cause the system to perform additional operations comprising: identifying a first insurance code identifier of the subset of the plurality of insurance code identifiers; determining that the first insurance code identifier corresponds to a first format of insurance code identifiers; and modifying the first insurance code identifier to produce a second insurance code identifier that corresponds to a second format of insurance code identifiers.

Aspect 36. The one or more non-transitory computer-readable media of aspect 35, comprising additional computer-executable instructions that, when executed by the one or more hardware processing units, cause the system to perform additional operations comprising: generating one or more additional requests of an additional API that include the second insurance code identifier; and obtaining, in response to the one or more additional calls of the additional API, an additional data file that includes information corresponding to the third insurance code identifier; and extracting the insurance code identifier from the additional data file.

Aspect 37. The one or more non-transitory computer-readable media of any one of aspects 31-34, comprising additional computer-executable instructions that, when executed by the one or more hardware processing units, cause the system to perform additional operations comprising: generating one or more additional calls of the API that include an additional insurance code identifier; obtaining, in response to the one or more additional calls of the API, an additional data file that includes additional information corresponding to the additional insurance code identifier; and determining that at least one valid insurance code identifier is not included in the additional information.

Aspect 38. The one or more non-transitory computer-readable media of any one of aspects 31-37, comprising additional computer-executable instructions that, when executed by the one or more hardware processing units, cause the system to perform additional operations comprising: generating one or more additional calls of the API that include an additional insurance code identifier of the subset of the plurality of insurance code identifiers; obtaining, in response to the one or more additional calls of the API, an additional data file that includes additional information corresponding to the additional insurance code identifier; analyzing the additional information with respect to one or more additional criteria; and determining that the additional information does not include at least one drug identifier.

Aspect 39. The one or more non-transitory computer-readable media of any one of aspects 31-38, comprising additional computer-executable instructions that, when executed by the one or more hardware processing units, cause the system to perform additional operations comprising: generating one or more additional calls of an additional API that includes the treatment identifier; obtaining, in response to the one or more additional calls, an additional data file that includes additional information corresponding to the drug identifier; and determining, based on the additional information, a class of drugs that corresponds to the drug identifier.

Aspect 40. The one or more non-transitory computer-readable media of any one of aspects 31-39, comprising additional computer-executable instructions that, when executed by the one or more hardware processing units, cause the system to perform additional operations comprising: determining, based on the additional information, a source of the class of drugs analyzing the source of the class of drugs with respect to a prioritized number of drug sources; extracting, from one or more fields of the data file, at least a portion of the additional information; and adding the at least a portion of the additional information to the row of the database table.

Aspect 41. The one or more non-transitory computer-readable media of aspect 40, wherein the prioritized number of drug sources includes a first source of the class of drugs having a first priority and a second source of the class of drugs having a second priority that is lower than the first priority.

Aspect 42. The one or more non-transitory computer-readable media of aspect 40 or 41, comprising additional computer-executable instructions that, when executed by the one or more hardware processing units, cause the system to perform additional operations comprising: analyzing the additional information with respect to the first source of the class of drugs; determining that the additional information does not include a source of the class of drugs that corresponds to the first class; analyzing the additional information with respect to the second sources of the class of drugs; and extracting at least a portion of the additional information from the additional data file in relation to the second source of the class of drugs.

Aspect 43. The one or more non-transitory computer-readable media of any one of aspects 31-42, comprising additional computer-executable instructions that, when executed by the one or more hardware processing units, cause the system to perform additional operations comprising: receiving a request to identify a group of individuals that received a treatment in response to a biological condition being present with respect to the group of individuals, wherein the request includes a name of the treatment or a class of the treatment; analyzing one or more values of one or more rows of the additional data table to determine one or more insurance code identifiers that correspond to the name of the treatment or the class of the treatment; analyzing values of a number of rows of the one or more data tables to determine one or more rows that include the one or more insurance code identifiers; and determining one or more identifiers of individuals included in the one or more rows to produce a cohort of individuals that received the treatment in relation to the biological condition.

Aspect 44. The one or more non-transitory computer-readable media of aspect 43, wherein the one or more computer-readable storage media store additional computer-executable instructions that, when executed by the one or more hardware processing units, cause the system to perform additional operations comprising: determining genomics information of the cohort of individuals that received the treatment in relation to the biological condition; and applying at least one of one or more statistical techniques or one or more machine learning techniques to determine one or more features of the cohort of individuals.

Aspect 45. The one or more non-transitory computer-readable media of aspect 44, wherein the one or more features include at least one of a genetic mutation included in respective genomes of individuals included in the cohort of individuals, a genetic mutation of cell-free deoxyribonucleic acids (DNA) included in one or more samples obtained from individuals included in the cohort of individuals, an amount of cell-free DNA having the genetic mutation for the respective individuals included in the cohort of individuals, or a change in the amount of cell-free DNA having the genetic mutation for the respective individuals included in the cohort of individuals over a period of time.

As used herein, a component, can refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

It should be understood that the individual steps used in the methods of the present teachings may be performed in any order and/or simultaneously, as long as the teaching remains operable. Furthermore, it should be understood that the apparatus and methods of the present teachings can include any number, or all, of the described implementations, as long as the teaching remains operable.

The various steps of the methods disclosed herein, or the steps carried out by the systems disclosed herein, may be carried out at the same time or different times, and/or in the same geographical location or different geographical locations, e.g., countries. The various steps of the methods disclosed herein can be performed by the same person or different people.

Various implementations of systems, devices, and methods have been described herein. These implementations are given only by way of example and are not intended to limit the scope of the claimed inventions. It should be appreciated, moreover, that the various features of the implementations that have been described may be combined in various ways to produce numerous additional implementations. Moreover, while various materials, dimensions, shapes, configurations and locations, etc. have been described for use with disclosed implementations, others besides those disclosed may be utilized without exceeding the scope of the claimed inventions.

Persons of ordinary skill in the relevant arts will recognize that implementations may comprise fewer features than illustrated in any individual implementation described above. The implementations described herein are not meant to be an exhaustive presentation of the ways in which the various features may be combined. Accordingly, the implementations are not mutually exclusive combinations of features; rather, implementations can comprise a combination of different individual features selected from different individual implementations, as understood by persons of ordinary skill in the art. Moreover, elements described with respect to one implementation can be implemented in other implementations even when not described in such implementations unless otherwise noted. Although a dependent claim may refer in the claims to a specific combination with one or more other claims, other implementations can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one implementation,” “an implementation,” or “some implementations” means that a particular feature, structure, or characteristic, described in connection with the implementation, is included in at least one implementation of the teaching. The appearances of the phrase “in one implementation” in various places in the specification are not necessarily all referring to the same implementation.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Although an implementation has been described with reference to specific example implementations, it will be evident that various modifications and changes may be made to these implementations without departing from the broader spirit and scope of the present disclosure. Accordingly, the specification and drawings are to be regarded in an illustrative rather than a restrictive sense. The accompanying drawings that form a part hereof show, by way of illustration, and not of limitation, specific implementations in which the subject matter may be practiced. The implementations illustrated are described in sufficient detail to enable those skilled in the art to practice the teachings disclosed herein. Other implementations may be utilized and derived therefrom, such that structural and logical substitutions and changes may be made without departing from the scope of this disclosure. This Detailed Description, therefore, is not to be taken in a limiting sense, and the scope of various implementations is defined only by the appended claims, along with the full range of equivalents to which such claims are entitled.

Although specific implementations have been illustrated and described herein, it should be appreciated that any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, user equipment (UE), article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

1. A method comprising:
analyzing, by a computing system including processing circuitry and memory, one or more data tables stored by a data repository with respect to one or more criteria, the one or more data tables including insurance claims data corresponding to one or more treatments provided to an individual for a biological condition and the one or more criteria indicating one or more formats of insurance code identifiers;
determining, by the computing system, a plurality of insurance code identifiers included in the one or more data tables that corresponds to the one or more criteria,
analyzing, by the computing system, a subset of the plurality of insurance code identifiers to determine one or more formats of the subset of the plurality of insurance code identifiers;
generating, by the computing system, one or more requests of an application programming interface (API) calls that include an insurance code identifier of the subset of the plurality of insurance code identifiers;
obtaining, by the computing system and in response to the one or more requests of the API, a data file that includes information corresponding to the insurance code identifier;
extracting, by the computing system, a treatment identifier from the data file; and
generating, by the computing system, an additional data table with a row indicating that the insurance code identifier corresponds to the treatment identifier.

2. The method of claim 1, comprising determining, by the computing system, one or more columns of the one or more data tables that include insurance code identifiers corresponding to treatment of individuals.

3. The method of claim 1, comprising:
determining, by the computing system, that a first insurance code identifier of the plurality of insurance code identifiers is a duplicate of a second insurance code identifier of the plurality of insurance code identifiers; and
removing, by the computing system, the second insurance code identifier from the plurality of insurance code identifiers to produce the plurality of insurance code identifiers.

4. The method of claim 1, wherein the plurality of insurance code identifiers correspond to a plurality of National Drug Code (NDC) identifiers.

5. The method of claim 1, comprising:
identifying, by the computing system, a first insurance code identifier of the subset of the plurality of insurance code identifiers;
determining, by the computing system, that the first insurance code identifier corresponds to a first format of insurance code identifiers; and
modifying, by the computing system, the first insurance code identifier to produce a second insurance code identifier that corresponds to a second format of insurance code identifiers.

6. The method of claim 5, comprising:
generating, by the computing system, one or more additional requests of an additional API that include the second insurance code identifier;
obtaining, by the computing system and in response to the one or more additional calls of the additional API, an additional data file that includes information corresponding to the third insurance code identifier; and
extracting, by the computing system, the insurance code identifier from the additional data file.

7. The method of claim 1, comprising:
generating, by the computing system, one or more additional calls of the API that include an additional insurance code identifier;
obtaining, by the computing system and in response to the one or more additional calls of the API, an additional data file that includes additional information corresponding to the additional insurance code identifier; and
determining, by the computing system, that at least one valid insurance code identifier is not included in the additional information.

8. The method of claim 1, comprising:
generating, by the computing system, one or more additional calls of the API that include an additional insurance code identifier of the subset of the plurality of insurance code identifiers;
obtaining, by the computing system and in response to the one or more additional calls of the API, an additional data file that includes additional information corresponding to the additional insurance code identifier;
analyzing, by the computing system, the additional information with respect to one or more additional criteria; and
determining, by the computing system, that the additional information does not include at least one drug identifier.

9. The method of claim 1, comprising:
generating, by the computing system, one or more additional calls of an additional API that includes the treatment identifier;
obtaining, by the computing system and in response to the one or more additional calls, an additional data file that includes additional information corresponding to the drug identifier; and
determining, by the computing system and based on the additional information, a class of drugs that corresponds to the drug identifier.

10. The method of claim 1, comprising:
determining, by the computing system and based on the additional information, a source of the class of drugs
analyzing, by the computing system, the source of the class of drugs with respect to a prioritized number of drug sources;
extracting, by the computing system and from one or more fields of the data file, at least a portion of the additional information; and
adding, by the computing system, the at least a portion of the additional information to the row of the database table.

11. The method of claim 10, wherein the prioritized number of drug sources includes a first source of the class of drugs having a first priority and a second source of the class of drugs having a second priority that is lower than the first priority;

12. The method of claim 10, comprising:
analyzing, by the computing system, the additional information with respect to the first source of the class of drugs;
determining, by the computing system, that the additional information does not include a source of the class of drugs that corresponds to the first class;
analyzing, by the computing system, the additional information with respect to the second sources of the class of drugs; and
extracting, by the computing system, at least a portion of the additional information from the additional data file in relation to the second source of the class of drugs.

13. The method of claim 1, comprising:
receiving, by the computing system, a request to identify a group of individuals that received a treatment in response to a biological condition being present with respect to the group of individuals, wherein the request includes a name of the treatment or a class of the treatment;
analyzing, by the computing system, one or more values of one or more rows of the additional data table to determine one or more insurance code identifiers that correspond to the name of the treatment or the class of the treatment;
analyzing, by the computing system, values of a number of rows of the one or more data tables to determine one or more rows that include the one or more insurance code identifiers; and
determining, by the computing system, one or more identifiers of individuals included in the one or more rows to produce a cohort of individuals that received the treatment in relation to the biological condition.

14. The method of claim 13, comprising:
determining, by the computing system, genomics information of the cohort of individuals that received the treatment in relation to the biological condition; and
applying, by the computing system, at least one of one or more statistical techniques or one or more machine learning techniques to determine one or more features of the cohort of individuals.

15. The method of claim 14, wherein the one or more features include at least one of a genetic mutation included in respective genomes of individuals included in the cohort of individuals, a genetic mutation of cell-free deoxyribonucleic acids (DNA) included in one or more samples obtained from individuals included in the cohort of individuals, an amount of cell-free DNA having the genetic mutation for the respective individuals included in the cohort of individuals, or a change in the amount of cell-free DNA having the genetic mutation for the respective individuals included in the cohort of individuals over a period of time.

16. A system comprising:
one or more hardware processing units;
one or more computer-readable storage media storing computer-executable instructions that, when executed by the one or more hardware processing units, cause the system to perform operations comprising:
analyzing one or more data tables stored by a data repository with respect to one or more criteria, the one or more data tables including insurance claims data corresponding to one or more treatments provided to an individual for a biological condition and the one or more criteria indicating one or more formats of insurance code identifiers;
determining a plurality of insurance code identifiers included in the one or more data tables that corresponds to the one or more criteria,
analyzing a subset of the plurality of insurance code identifiers to determine one or more formats of the subset of the plurality of insurance code identifiers;
generating one or more requests of an application programming interface (API) calls that include an insurance code identifier of the subset of the plurality of insurance code identifiers;
obtaining, in response to the one or more requests of the API, a data file that includes information corresponding to the insurance code identifier;
extracting a treatment identifier from the data file; and
generating an additional data table with a row indicating that the insurance code identifier corresponds to the treatment identifier.

17-30. (canceled)

31. One or more non-transitory computer-readable storage media storing computer-executable instructions that, when executed by one or more hardware processing units, cause a system to perform operations comprising:
analyzing one or more data tables stored by a data repository with respect to one or more criteria, the one or more data tables including insurance claims data corresponding to one or more treatments provided to an individual for a biological condition and the one or more criteria indicating one or more formats of insurance code identifiers;
determining a plurality of insurance code identifiers included in the one or more data tables that corresponds to the one or more criteria,
analyzing a subset of the plurality of insurance code identifiers to determine one or more formats of the subset of the plurality of insurance code identifiers;
generating one or more requests of an application programming interface (API) calls that include an insurance code identifier of the subset of the plurality of insurance code identifiers;
obtaining, in response to the one or more requests of the API, a data file that includes information corresponding to the insurance code identifier;
extracting a treatment identifier from the data file; and
generating an additional data table with a row indicating that the insurance code identifier corresponds to the treatment identifier.

32. The one or more non-transitory computer-readable media of claim 31, comprising additional computer-executable instructions that, when executed by the one or more hardware processing units, cause the system to perform additional operations comprising determining one or more columns of the one or more data tables that include insurance code identifiers corresponding to treatment of individuals.

33. The one or more non-transitory computer-readable media of claim 31, comprising additional computer-executable instructions that, when executed by the one or more hardware processing units, cause the system to perform additional operations comprising:
determining that a first insurance code identifier of the plurality of insurance code identifiers is a duplicate of a second insurance code identifier of the plurality of insurance code identifiers; and
removing the second insurance code identifier from the plurality of insurance code identifiers to produce the plurality of insurance code identifiers.

34. The one or more non-transitory computer-readable media of claim 31, wherein the plurality of insurance code identifiers correspond to a plurality of National Drug Code (NDC) identifiers.

35. The one or more non-transitory computer-readable media of claim 31, comprising additional computer-executable instructions that, when executed by the one or more hardware processing units, cause the system to perform additional operations comprising:
identifying a first insurance code identifier of the subset of the plurality of insurance code identifiers;
determining that the first insurance code identifier corresponds to a first format of insurance code identifiers; and
modifying the first insurance code identifier to produce a second insurance code identifier that corresponds to a second format of insurance code identifiers.

36. The one or more non-transitory computer-readable media of claim 35, comprising additional computer-executable instructions that, when executed by the one or more hardware processing units, cause the system to perform additional operations comprising:
generating one or more additional requests of an additional API that include the second insurance code identifier; and
obtaining, in response to the one or more additional calls of the additional API, an additional data file that includes information corresponding to the third insurance code identifier; and
extracting the insurance code identifier from the additional data file.

37-45. (canceled)
